(19) United States
(12) Patent Application Publication
(10) Pub. No.: US 2025/0285714 A1
(43) Pub. Date: Sep. 11, 2025

(54) A PHARMACEUTICAL PLATFORM TECHNOLOGY FOR DRUG DISCOVERY AND CONSUMER HEALTH PRODUCT DEVELOPMENT

(71) Applicant: SINOVEDA CANADA INC., Alberta (CA)

(72) Inventors: Yun Kau TAM, ALBERTA (CA); Chih-Yuan TSENG, ALBERTA (CA); Trent BJORNDAHL, ALBERTA (CA)

(21) Appl. No.: 18/684,451

(22) PCT Filed: Aug. 19, 2022
(86) PCT No.: PCT/IB2022/057780

§ 371 Date: Feb. 16, 2024

Related U.S. Application Data
(60) Provisional application No. 63/234,917, filed on Aug. 19, 2021.
(60) Provisional application No. 63/234,910, filed on Aug. 19, 2021.

(51) Int. Cl. G16C 20/30; A61K 31/00; G16C 20/20; G16C 20/70

(52) U.S. Cl. CPC G16C 20/30; A61K 31/658; G16C 20/20; G16C 20/70

(57) ABSTRACT

In one embodiment, the present invention describes a method for identifying an optimized natural medicine containing defined doses of active and contributing ingredients. In one embodiment, the method disclosed in the present invention develops compositions comprising cannabinoids for the treatment of hepatocellular carcinoma.

### FIELD OF THE INVENTION

The present invention relates to a platform technology which engages pharmaceutical design methodologies to mine and quantify active and contributing ingredients in an herbal formula like that of Traditional Chinese Medicine.

Throughout this application, various references are referred to and disclosures of these publications in their entireties are hereby incorporated by reference into this application to more fully describe the state of the art to which this invention pertains.

### BACKGROUND OF THE INVENTION

Traditional medicine including Traditional Chinese Medicine (TCM) has been used for thousands of years. Most of these traditional medicinal formulas consist of either plant-, animal- or mineral-based formulas. These formulas are designed to treat specific conditions that are unique to individuals who have been diagnosed using traditional paradigms. Unlike conventional pharmaceuticals, the aims of deploying TCM are to treat diseases and restore the patient to a state of balance.

TCM has been gaining popularity around the world. In 2018, Hong Kong South China Morning Post reported that the size of TCM market approached $50 billion USD globally (https://www.scmp.com/news/china/society/article/2166278/traditional-chinese-medicine-closes-us50-billion-market-long.)

As a country, China has invested a lot of resources to promote and advance the research of TCM. In the 13th Five-year plan, the State Council of China has mandated modernization of TCM (KPMG, 2016, The 13th Five-Year Plan-China's transformation and integration with the world economy). Early in 2016, the Chinese government published a blueprint for developing TCM over the next 15 years. TCM, it said, should have equal status in law with modern medicine, and should be regulated as such (https://www.economist.com/news/china/21727945-unproven-remedies-promoted-state-why-chinas-traditional-medicine-boom-dangerous). In the later part of 2016, a “white-paper” was issued saying that TCM would play a big role in reforming the health-care system because of its relatively low cost (http://english.gov.cn/archive/white paper/2016/12/06/content_28 1475509333700.htm) (Wang, Zhou et al. 2021).

Traditional medicine, including TCM, reached a new milestone in April 2019. In the 11th version of World Health Organization's (WHO) International Statistical Classification of Diseases and Related Health Problems (ICD), Chapter 26, traditional remedies are listed for the first time.

Despite the popularity of TCM and Chinese government support, main-stream scientific, medical, and pharmaceutical communities have been highly skeptical and critical of the therapeutic values of TCM. This criticism is largely due to the complexity of TCM in terms of identity and quantity of active constituents, hence, consistency and quality, limited understanding of mechanisms of action, and lack of clinical evidences that have been subjected through the rigor of pharmaceutical trials (Graziose, Lila et al. 2010) (https://www.scientificamerican.com/article/the-world-health-organization-gives-the-nod-to-traditional-chinese-medicine-bad-idea/?redirect=1). It is quite apparent that the pharmacological, clinical, and quality gaps between TCM and modern medicine need to be bridged before traditional medicine is fully accepted by the mainstream. This void creates an unmet need for research in this area.

To address the knowledge gap between TCM and modern medical sciences, it is crucial to understand the structure of a TCM formula. A TCM formula composes of four major types of ingredients; they are Jun (emperor, principle active ingredients, a must have in the formula), Chen (assistant to the emperor, through enhancement of the actives), Zuo (facilitator in ways of reducing secondary symptoms or toxicity of the actives) and Shi (guide the actives to the sites of action).

In China and around the world, there is a drive to link the concept of TCM formulation to mechanisms of drug action, the way modern science is built upon. A few approaches such as data mining, in silico modelling and predictions, systems biology, multiomics, genetic, bioinformatics, network pharmacology and quality markers have been used to study TCM formulas. There are successful examples in identifying active ingredients and their mechanisms of action (Jiang, Zhang et al. 2012, Liang, Jiang et al. 2012, Shi, Zhao et al. 2012, Sun, Dai et al. 2012, Zhang, Li et al. 2013, Su, Jia et al. 2014, Dai 2019, Zhao, Liu et al. 2019), however, the therapeutic values of these components, acting individually or in combination have not been tested fully clinically.

TCM scientists are cognizant that active ingredients must have appropriate drug-like properties, in other words, suitable pharmacokinetic properties, to be therapeutically active. Attempts have been made to narrow the list of actives down to those having appropriate drug-like properties. Unfortunately, the pharmacokinetic properties of these ingredients are usually derived from animals (Wang, Sun et al. 2011), which may have limited application to humans. A rapid and accurate method for predicting human pharmacokinetic properties of a test compound is desired.

Furthermore, the contribution by active metabolites and their drug-like properties are seldom accounted for in these studies. Metabolism of herbal constituents occur frequently in the gut lumen, mainly by the microbiome, intestinal cells and the liver. A famous example is Panax ginseng, the ginsenosides, by themselves are not active, their colonic metabolites like compound K, are (Hasegawa 2004). In terms of active metabolites, the site of production adds complexity for the quantification of these compounds, particularly metabolites produced by the microbiome.

As TCM pharmacology research advances, it is recognized that, to successfully develop a pharmaceutical with attributes of a TCM formula, a few hurdles must be overcome, since the final product contains multiple components addressing various issues in the body.

Bioinformatics, systems biology, network pharmacology, data mining, in silico modeling including docking are great tools for identifying potential active ingredients. This is an initial step for both conventional drug discovery and TCM research. Where the two diverge is the former is looking for a single compound, and the latter is aiming at a group of compounds, working on numerous targets in networks that may or may not be associated.

These in silico exercises furnish a direction for in vitro research. A major complication of TCM research is the number of studies and samples involved, high throughput processing is often engaged. Especially when multiomics and network pharmacological methods are incorporated into the protocols.

While the workflows of conventional drug discovery and TCM development are similar, due to the complexity of a TCM formula, which contains multiple herbs, and each herb could easily contain more than 1000 compounds, a different strategy would have to be devised.

Before a methodology is designed to develop a pharmaceutical while maintaining the characteristics of TCM, a ‘translation’ of the TCM formula design in terms of modern sciences would be necessary. Furthermore, a few issues would have to be identified, tackled, and included in the method.

Jun, the emperor, denotes active ingredients; Chen, are ingredients that are less active and/or could interact with the actives to enhance efficacy; Zou, are ingredients which are inhibitors of Jun's toxic activity; and Shi, are ingredients which guide the actives to the site of action. In this design, there are actives (Jun); there are components that are less active, but they either act on the same receptors or pathways/networks that enhance the effects of the actives (Chen); there are compounds that treat symptoms of a disease or inhibit the toxicity of an active (Zou); and there are compounds that guide the actives to the site of action or change its fate in the body, such as pharmacokinetics (Shi).

The ingredients, if they contribute to the overall activity of the formula, do not have to be active. Interactions among ingredients could be active-active, active-inactive, or potentially inactive-inactive. Taking these features into account, the developmental process is not remotely close to that employed in conventional pharmaceuticals.

The pharmacokinetic properties of active ingredients, for example, artemisinin, were improved by constituents in A. annua (Weathers, Elfawal et al. 2014, Weathers, Towler et al. 2014). This example is a good illustration of a ‘Shi’ candidate. Conventional screening process would not target components without activities.

Tam et al. (2019) have shown that genistein, a minute and less active ingredient potentiates the activity of the principle ingredient, Biochanin A. In return, Biochanin A counteracts potential toxicity of genistein. In one extract, there exists Jun, Chen and Zou.

In multiple-ingredient product development, the conventional wisdom is that two body interactions are the prime reaction between molecules. Higher order interactions are assumed to have negligible contributions. A recent publication in yeast clearly showed that higher order interactions in a biological system have significant contributions (Tekin, White et al. 2018). When higher order interactions are considered, the burden to tease out these contributions is onerous, and it would render the pursuit impractical (FIG. 1). Potentially, billions of data points might be required, and the sampling load would increase when multiple targets are involved. It is apparent that there is a need to simplify the process of evaluating higher order interactions.

Although the mechanistic understanding of TCM is deepened using modern sciences, it is not surprising that there are no pharmaceutical products derived from traditional TCM formulas that contain ingredients with attributes pertaining to the four pillars of TCM formulation: Jun, Chen, Zou and Shi.

Using an integrated approach, Tam & Tuszynski (2008) have successfully developed a pharmaceutical platform technology (PPT) to identify and quantify active ingredients from a complex mixture, similar to that present in medicinal herbs.

This technique has been applied to identify contributing components in botanicals, which have been tested clinically. Unlike the conventional pharmaceutical approach, whose aim is to search for the most potent chemical entity, the aim of using PPT is to reveal a group of active/contributing ingredients, which is responsible for their pharmacological actions.

The inventors reckoned that botanical pharmaceuticals developed using PPT would have a higher chance of success because they have a long history of use and quite frequently, there are supporting clinical data.

The approach of Tam and Tuszynski's (2008) requires randomization of unknown substances in a complex mixture. This whole process, from the beginning to the end, may require hundreds of thousands of samples (FIG. 1(?)).

In this invention, a simplified and improved method for identifying and quantifying active and contributing ingredients in an herb or herbal formula is described.

### SUMMARY OF THE INVENTION

In one embodiment, the present invention discloses a pharmaceutical platform technology (generation II) (PPT-II) which engages drug discovery methodologies to mine and quantify active and contributing ingredients in an herbal formula like that of Traditional Chinese Medicine. These ingredients are responsible for and consistent with the paradigm of TCM or other disciplines of herbal medicines.

In one embodiment, in silico methodologies are used to screen herbs or herbal formulas. The intent is to rapidly arrive at the number of compounds of interest.

In one embodiment, compounds of interest are subject to in vitro screen to confirm their likelihood of having efficacy and proper drug-like properties.

In one embodiment, compounds of interest are screened with specific in vitro models along with in silico strategies.

In one embodiment, pharmacokinetic and pharmacodynamic properties of compounds are validated in vitro.

In one embodiment, the ratios of compounds of interest are optimized before the declaration of a lead.

In one embodiment, systems biology, systems pharmacology, and bioinformatics are incorporated into the present invention to enhance the probability of identifying constituents that are responsible for the clinical response of the botanical mixture.

In one embodiment, machine learning based approaches are embedded in the workflow of PPT-II to assist in data mining and analysis, and the choice of preclinical models and their clinical relevance for the validation of the identified candidates. In another embodiment, the choice of potential actives is backed by mechanisms of action.

In one embodiment, Cluster expansion method is engaged to model multi-body interactions among contributing ingredients.

In one embodiment, the method described in the present invention is used to develop compositions of products derived from cannabis for the treatment of liver cancer.

In one embodiment, the cannabis derived compositions consist of two to three active moieties. In another embodiment, the compositions work additively or synergistically together.

### BRIEF DESCRIPTION OF THE FIGURES

FIG. 1 shows the relationship between the number of interactants (N) and the number of interactions required to evaluate multiple body interactions.

FIG. 2 shows the workflow of PPT-II.

FIG. 3 describes Step 2 of the PPT-II flowchart (PD=pharmacodynamics, PK=pharmacokinetics, MTII=metabolic and transporter inducer or inhibitor, and * repeats of Step 2.1 where there could be more than two compounds).

FIG. 4 shows the integrated platform of PPT-II.

FIG. 5 shows an image of a 3D HepG2 spheroid.

FIG. 6 shows the relationship between oral bioavailability (Fb) and systemic exposure, area under the plasma concentration vs. time curve (AUC). Insets a. Cannabinoids with Fb 90% or lower; b. Cannabinoids with Fb 90% or higher.

FIG. 7 shows chemical structures of cannabinol (CBN), cannabidiol (CBD), and cannabichromene (CBC).

FIG. 8A shows comparison of IC₅₀ values obtained for CBD, CBC and CBN against HepG2 cells in IMDM media with varying concentrations of fetal bovine serum (FBS).

FIG. 8B shows a repeat of IC₅₀ experiment for CBC against HepG2 cells with an extended range of FBS concentrations.

FIG. 8C shows IC₅₀ values for CBD, CBC, and CBN reported relative to sorafenib observed in IMDM media containing 1%, 10% and 20% FBS.

FIG. 9 shows comparisons of observed IC₅₀ values for ternary ratios of CBD:CBC:CBN obtained for HepG2 cells in IMDM media containing 20% FBS, 1% ethanol. The CBN ratio is relative to the other cannabinoid components is 1.

FIG. 10A. Comparisons of IC₅₀ values of CBD, CBC, CBN, combinations there-of, and sorafenib conducted in IMDM media with 20% FBS and 1% ethanol (SCI-931: CBD:CBC:CBN 9:3:1; SCI-421: CBD:CBC:CBN 4:2:1; SCI-111: CBD:CBC:CBN 1:1:1).

FIG. 10B. Comparison of IC50 values for three ternary combinations of CBD:CBC:CBN against HepG2 cells in media with varying amounts of FBS. Legend numbers correspond to ratio values.

FIG. 11A. CBD:CBC:CBN 9:3:1 IC₅₀ plot for HepG2 spheroids conducted in DMEM media with 1% FBS after 48 hours.

FIG. 11B. Phase contrast (top) and fluorescent (bottom) microscopy images of HepG2 spheroids treated with combination of CBD:CBC:CBN (9:3:1) for 48 hours. Fluorescent images obtained after a one-hour incubation at 37° C. with CyQuant™.

### DETAILED DESCRIPTION OF THE INVENTION

This invention discloses a set of specifically designed procedures consisting of a blend of in silico and in vitro methodologies, permitting the deciphering of active and related compounds from a botanical or natural formula. The uniqueness of this methodology is that it provides a means to develop a multi-component drug or a consumer health product candidate in a short time span with a high probability of clinical success.

In one embodiment, in silico methodologies comprise systems biology, systems pharmacology and bioinformatics for data mining and data analysis.

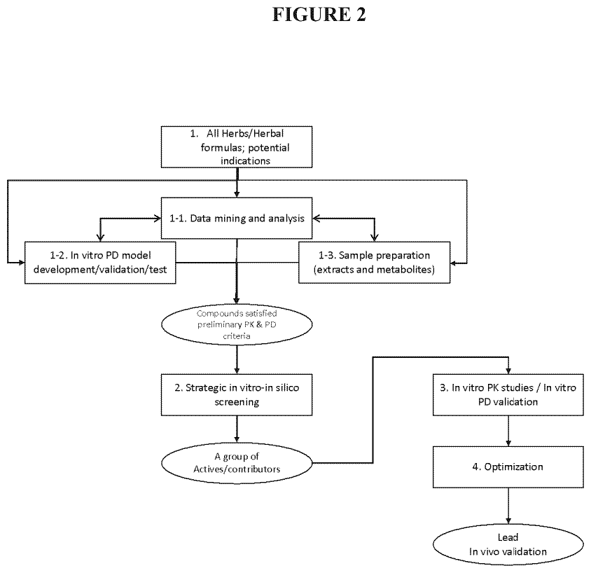

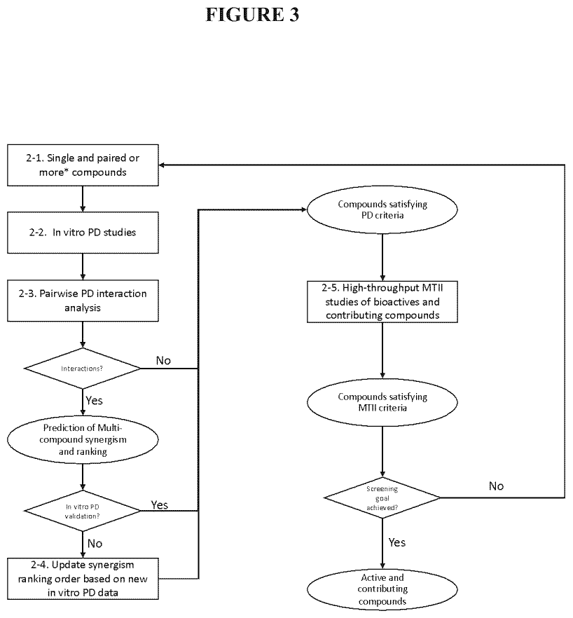

In one embodiment, machine learning based approaches are used in different areas of the procedures to enhance the thoroughness of data mining (Step 1-1 of FIG. 2) and the accuracy of strategic screening (Step 2-3 of FIG. 3), to deduce potential mechanisms of action of bioactives in a disease network, or to choose appropriate in vitro models for providing clinically relevant results (Step 1-2, FIG. 2).

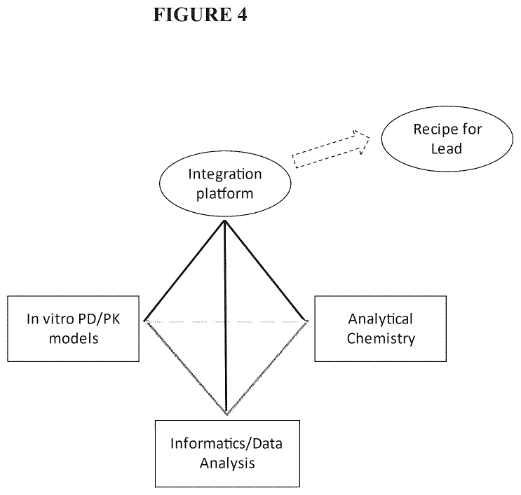

In one embodiment, the structure of the PPT-II's research module described in this invention contains three interacting units managed by the control center (Computational Unit, FIG. 4); they are the bioinformatics, in vitro and analytical units. The computational unit serves as the brain, the bioinformatics unit is responsible for data mining, data analysis, and modeling and simulations, providing insights into the feasibility of a potential project. The in vitro and analytical units receive instructions, in terms of experimental design, and in return, data for processing and analysis. There is also a direct communication between the analytical and the in vitro unit since the analytical group will supply samples to the in vitro unit for PK and PD measurements.

The flow charts shown in FIGS. 2 and 3 describe the workflow of the whole process. The validity of each of the step described in these schematics will be demonstrated in Examples.

In one embodiment, the main goal of this invention is to establish a set of clinically relevant parameters to describe the efficacies and drug-like properties of active and contributing constituents in an herbal formula.

In one embodiment, the processes described in this invention are designed to rapidly and efficiently discard extraneous components which have no or very limited contributions to the overall effects of an herbal formula (FIGS. 2 and 3).

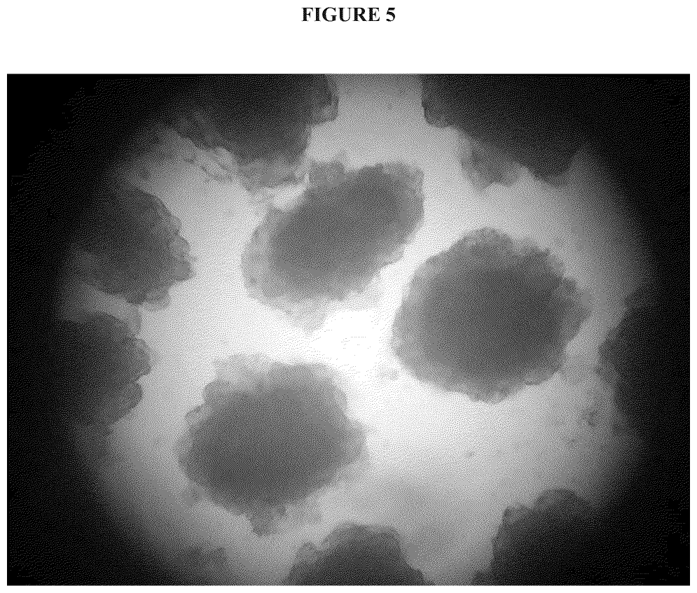

In one embodiment, in vitro models, for example, 3D cell culture (FIG. 5), organoids, patient derived xenografts (PDXs), cells on a chip (CoC) are employed to mimic disease conditions in patients in vitro.

In one embodiment, appropriate in vitro models are established to estimate efficacy, or pharmacodynamics (PD), or absorption (A), distribution (D), metabolism (M), excretion (E) toxicity (T) or PK of individual ingredients in an herb or herbal formula.

In one embodiment, key biomarkers of an in vitro model, known and unknown, will be selected from a few signaling pathways using the newly designed high throughput system.

In one embodiment, these markers will be used to quantify the efficacy of active and contributing ingredients.

In one embodiment, the absorption of individual ingredients is evaluated using either the traditional CaCO-2 or MDCK cell models. Alternatively, a 3D intestinal model, for example, organ-on-a-chip model will be engaged to estimate the extent of absorption of herbal compounds.

In one embodiment, metabolism in the enterocytes will be estimated using human intestinal microsomes. Alternatively, a 3D intestinal model, for example, organ-on-a-chip model will be engaged to estimate in vivo intestinal metabolism.

In one embodiment, luminal stability of an ingredient will be estimated by incubating an ingredient with simulated gastric or intestinal fluids, mimicking the fast and fed state.

In one embodiment, fecal metabolism of an ingredient will be estimated using an established anerobic method.

In one embodiment, hepatic metabolism of an ingredient is evaluated using either, human liver microsomes, S-9 fraction, hepatocytes or a 3D model of a human liver.

In one embodiment, excretion of an ingredient is estimated using an established in silico method. Alternatively, a 2D kidney model, such as a monolayer of MDCK cells, or a 3D model organoid model will be used to estimate renal excretion of an ingredient.

In one embodiment, the distribution of an ingredient will be estimated using an in vitro method reported by Mayumi, Tachibana et al. (2020). The volume of distribution and the profile of actives at the site of action will be estimated by incorporating in vitro measurements of partition into the organ.

In one embodiment, the pharmacokinetic parameters of the possible actives and MTII will be estimated using the in vitro models established in this invention.

In one embodiment, the PD and PK parameters estimated in vitro will be scaled to a human physiologically based pharmacodynamic and pharmacokinetic (PBPKPD) model.

In one embodiment, dosages of active and contributing ingredients will be calculated to achieve optimum concentrations at the sites of action using the parametrized PBPKPD model.

Based on model prediction, appropriate routes of administration and types of dosage forms will be determined to achieve optimized time course of actives at the sites of action.

In one embodiment, the present invention provided a method to efficiently identify a composition comprising active compounds from an herb or herbal formula for treating a disease, said method comprising:
1) Obtaining chemical profile in an herb or herbal formula from existing databases using data mining and machine learning algorithms or producing chemical profile using high resolution mass spectrometry if no records are found in existing databases;
2) Identifying and developing appropriate in vitro models associated with the disease of interests that have clinical relevance;
3) Computationally identify, from the chemical profile obtained in step 1), potential active ingredients as candidate methods compounds using from systems biology, systems pharmacology, bioinformatics and machine learning based approaches based on three criteria,
   a. Their efficacies in treating the disease;
   b. Their influence on the absorption and metabolism of potential active ingredients;
   c. Their drug-like properties and their metabolites;
4) Strategically screen the list of primary candidate compounds obtained in step 3) by following a set of predetermined and adjustable pharmacodynamic and pharmacokinetic criteria based on a pairwise-based experimental approach using in vitro models developed in step 2), resulting in a list of secondary candidate compounds, and
5) Validating and testing the secondary candidate compounds for efficacy and side effects in vitro, leading to a list of active compounds; and
6) Formulating a composition comprising the active compounds in view of compound-compound interactions, wherein the composition possesses a maximum efficacy with minimum side effects in treating the disease.

In one embodiment, the chemical profile in an herb or herbal formula refers to a list of chemical substances in the herb or herbal formula. In one embodiment, the chemical profile comprises chemicals metabolized or derived from the chemical substances in the herb or herbal formula. In one embodiment, the chemical profile comprises chemicals that could induce or inhibit metabolism of or alter the transport of certain compounds in vitro or in vivo.

In one embodiment, the compound-compound interactions comprise pair-wise interactions. In one embodiment, the pair-wise interactions are quantified by two-dimensional arrays based on an in vitro model.

In one embodiment, the compound-compound interactions comprise higher order interactions. In one embodiment, the higher order interactions are predicted by using Cluster expansion using data from pair-wise interactions validated with experimental inputs.

In one embodiment, the drug-like properties are estimated using in vitro and in silico parameters as input for the physiologically based pharmacokinetic models.

In one embodiment, the drug-like properties generated in vitro and in silico are used to estimate in vivo pharmacokinetic parameters with proper scaling using one or more of the followings:
1) human intestinal microsomes;
2) 3D intestinal model;
3) simulated gastric or intestinal fluids;
4) established anerobic method;
5) human liver microsomes;
6) S-9 fraction;
7) hepatocytes;
8) 3D model of a human liver;
9) 2D kidney model;
10) 3D organoid model; and
11) Organ-on-a-chip model.

In one embodiment, the drug-like properties are estimated using existing algorithms, commercial or open-source software.

In one embodiment, the chemical profile in the herb or herb formula compounds that could induce or inhibit metabolism of or alter the transport of the potential active ingredients and the metabolites.

In one embodiment, the chemical profile in the herb or herbal formula comprises of compounds that could increase or decrease efficacies or side effects associated with one or more of the potential active ingredients and the metabolites in treating the disease.

In one embodiment, the composition is formulated in view of compound-compound interactions established using in vitro methods.

In one embodiment, the composition is formulated for easy or efficient delivery.

In one embodiment, the composition is formulated as tablets, solutions, suspensions, creams, emulsions, or nano encapsulated emulsions.

In one embodiment, the composition is formulated as a form for oral, sublingual, topical, subcutaneous, intramuscular, intravenous or intraarterial administration.

In one embodiment, the disease is selected from the group consisting of cancer, cardiovascular, hepatic, renal, lung, neurodegenerative, arthritic, immune, auto-immune diseases, and diseases which are described in TCM literature.

In one embodiment, the herb or herbal formula is or comprises cannabis or cannabinoids.

In one embodiment, the cannabinoids comprise of, but not limited to one or more of the followings:
1) cannabichromene (CBC);
2) cannabinol (CBN); and
3) cannabidiol (CBD).

The intent of the examples below is to describe the sequence of events involved and innovations of in vitro, in silico and analytical methods to improve parameter prediction and to enhance data mining and processing.

### Example 1

The objective of this example is to describe the integrative approach of this invention encompassing computation, in vitro and analytical processes designed to efficiently and accurately in generating a multiple-compound lead, which has the characteristics of an herbal formula (FIGS. 2 and 3).

After the decision is made to develop an herbal formula, a four-step approach is adopted to come up with a group of bioactives and contributing compounds which play critical roles in Jun, Chen, Zuo and Shi.

The first step is to use computational methodologies to screen the chemical profiles of the herbal formula for potentially active and contributing compounds, their metabolic pathways and formation of metabolites. Compounds, including metabolites, with appropriate ADME or drug-like properties will be included for further studies (Step 1 of FIG. 2).

Included in the first step is the use of in silico methodologies to predict metabolism of active compounds, and inactive compounds which could enhance or inhibit the metabolism of the bioactives. Potential significance of these compounds is depending on their PK properties, which will be calculated using software like ADME predictor.

Another part of the first step is to establish appropriate and standardized in vitro models and tools approved by US FDA to accurately describe disease processes (Step 1-2 of FIG. 2). The in vitro tools will be used to generate activity data of compounds, which are unavailable in databases, as part of in silico screening.

The second step is to perform strategic screening using a combination of one in vitro model or a system of in vitro models, analytical and in silico methodologies based on the framework of cluster expansion methods. The goals are to quantify efficacy of individual compounds and their potential interactions (Step 2, FIGS. 2 and 3).

Included in the second step, potential contributing compounds identified in the in-silico search with enzyme inducing or inhibiting properties are prepared and separated into 2 sets of samples, singled and paired compounds for further screening using standardized in vitro models approved or begin examined by US FDA (Step 2-1 and 2-2, FIG. 3).

Included in the second step is the use of a decision loop based on the cluster expansion based in silico model of compounds' potential interactions on in vitro PD responses to select compounds (Step 2-3, FIG. 3). If in vitro PD validation for higher order interactions does not agree with the predictions of the in-silico model, in vitro PD observed interactions will be used for refining the in-silico model and screening compounds in Step 2-4.

Included in the second step is the use of high-throughput MTII studies of compounds obtained from Step 2-3 or 2-4 (Steps 2-5, FIG. 3).

The third step is to use in vitro models designed to estimate human drug-like properties of the bioactives and contributing compounds (FIG. 2).

The fourth step is the optimization of the ratios of these compounds. The result is the generation of a lead for pre-clinical studies.

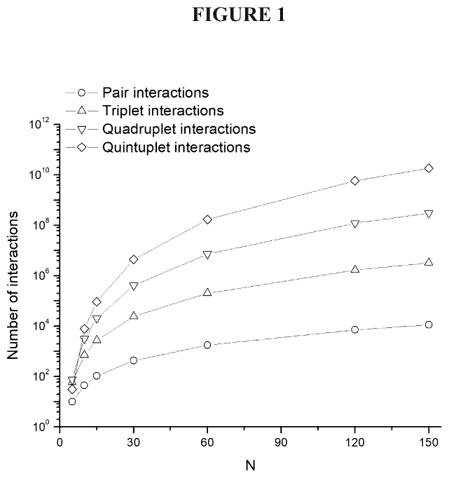

The unique features of this workflow are that:
1. No fractionation is required, as potential active and contributing compounds are identified in silico.
2. Potential active metabolites are identified early on. Since most of the TCMs are administered orally, and since a large percentage of naturally occurring compounds are in the glycoside forms, the real actives are the de-glycosylated, aglycone, forms. The number of aglycones is usually less than that of the corresponding glycosides.
3. The quantity of constituents is taken into consideration, when a specific compound is to be included in an analysis.
4. Higher order interactions are often neglected. The inclusion of this aspect of interaction would require astronomical number of samples (FIG. 1). Methodologies including, cluster expansion based on pairwise PD interaction studies and tools for data analysis including machine learning based non-linear regression, are used to lower the number of samples tested.
5. The incorporation of the above 4 points will drastically reduce the workload.

Compared to the conventional drug discovery approach, the speed of lead generation is higher, the risk is lower as the efficacy and toxicity of the herbal formula are mostly known, hence, the chance of success in clinical trials is highly enhanced. With defined contents and dosages of the lead, issues with quality control encountered by herbal medicines are overcome.

### Example 2

The objective of this example is to outline the computational processes described in this invention. Processes in place are described and processes to be developed are expounded.

Several algorithms and database management system have been developed to house TCM databases, data mining and analysis, dose-response and interaction analysis and cluster-expansion based interaction analysis.

A SQLite database management system was developed to store and organize data for chemicals, herbs, and disease networks generated from open-source databases or produced inhouse. A Graphic User Interface (GUI) was developed using PhP language for accessing the database. For chemicals, PubChem database from National Institutes of Health (https://pubchem.ncbi.nlm.nih.gov/) was used. For herbs, two TCM databases TM-MC (http://informatics.kiom.re.kr/compound/browse.do) and ETCM (http://www.tcmip.cn/ETCM/) were used. For disease networks, KEGG: Kyoto Encyclopedia of Genes and Genomes database (https://www.kegg.jp/) was used.

A prototype algorithm for data mining and analysis was designed using Python language with various bioinformatic and chemoinformatic packages to mine physiochemical, biological, and pharmacokinetic information of chemical of interests through PubChem database (https://pubchem.ncbi.nlm.nih.gov/) and signaling pathway information of disease of interests through KEGG (https://www.genome.jp/kegg/pathway.html). This algorithm is also equipped to work with several analysis tools which include basic descriptive statistical analysis, partition coefficient and minimal dose predictions, and chemical similarity comparison.

A prototype algorithm was designed for analyzing dose-response and compound-compound interactions. Python language was used to conduct cellular dose-response regressions for single and two compound mixtures.

A mathematical theory of cluster-expansion based interaction analysis was developed. The validity of this theory requires experimental data input for verification. This aspect of interactions considers higher order processes, which are missing in drug discovery.

An inhouse physiologically based pharmacokinetic (PBPK) simulation program has been established. It supports one, two and multi-compartment models. The compartmental model program was designed using Matlab and Simulink to emulate human physiological conditions. With appropriate human physiological parameter input, absorption, distribution, metabolism and excretion profiles of compounds of interest can be estimated in human. The current version supports both command-line and simple GUI operation (GUI requires Matlab 2014b or later).

The goal of the computational unit is to create an integrated workflow to seamlessly organize in vitro and analytical data accumulation, and computational databases and to efficiently screen compounds of interest as described in FIGS. 2 and 3. Lead information, PD/PK characteristics of the components in the lead, and the mechanisms of action form the template of data presentation. This workflow will become the backbone of PPT-II.

To achieve the integrated workflow, several areas require enhancements and new algorithms are needed to fulfill various aspects of computational screening and analytical process.

In the scope of data mining, the algorithm needs to be expanded to include databases like Reactome (https://reactome.org/) to evaluate protein-protein interaction, BioModels (https://www.ebi.ac.uk/biomodels/) which combine network and in vitro cellular reaction to provide a more wholesome understanding of a disease model, Genomics of Drug Sensitivity in Cancer (GDSC) database (https://www.cancerrxgene.org/) for signaling and clinical indications; this database has the potential to allow for in vitro and in vivo scaling in cancer treatment as it utilizes more than 1, 100 cancer cell lines for processing (Sakellaropoulos, Vougas et al. 2019), Zinc (https://zinc.docking.org/), a docking algorithm for ligand-protein interactions, and DrugBank (https://go.drugbank.com/) for FDA approved drugs, consisting of chemical, clinical, etc. information. This expansion will also include a more comprehensive PK and PD information for in silico evaluation. Furthermore, we will also explore the use of machine learning based natural language processing methods to data mine through literatures and web contents for both categories to enhance our own database.

Computational methods and tools to predict Phase I and II metabolisms for drug discovery (Tyzack and Kirchmair 2019) will be used to predict metabolism of compounds of interest.

Metabolic inhibitors are identified using methods published by Tyzack and Kirchmair (2019) and metabolic inducers will be identified using the method of Banerjee, Dunkel et al. (2020).

To enhance the accuracy of in vitro PD predictions in humans using gene expression data, machine learning algorithms published, and the database utilized by Sakellaropoulos, Vougas et al. (2019) and Geeleher, Cox et al. (2014) are used to fill the gap between in vitro models and humans.

To appropriately model higher order interactions based on in vitro PD data, which involve non-linear contributions from multiple-compound combinations, machine learning based non-linear regression methods will be used.

Two parts of the existing PBPK simulation program are improved. The first part is the introduction of better in vitro in vivo correlation methods to systematically improve in vitro scaling. The second part is the focus on developing computational fluid dynamic model to simulate dissolutions and absorptions of drugs in the GI tract.

### Example 3

Although in vitro assays described in the databases are suitable for the evaluation of the efficacy of a compound, it has limited utility for predicting physiological effective concentrations.

One of the main goals of this invention is to establish in vitro tools to accurately describe disease processes. 3-D cell models or organoids, and patient derived xenografts (PDx) have the features of an organ or tissue within the human body. In a disease condition, multiple pathways are affected across cell types. They are usually manifested in changes at the gene, protein, and marker levels. Single cell 2-D models, although useful in initial screening, would not provide information relevant to the whole disease process. For example, measurement of dopaminergic neuronal changes in Parkinson's Disease would not provide information of a leaky blood-brain-barrier and its effect on Parkinson's disease. Efficacies quantified using 3-D systems have a higher degree of accuracy in projecting into patients. This type of systems also allows the evaluation of multiple compounds' effects on multiple targets, a system that is most suitable for TCM research. Commercially available organoids and patient derived xenografts will be used unless they are not commercially available (https://www.corning.com/worldwide/en/products/life-sciences/resources/stories/in-the-field/glioma-stem-cells-enable-the-production-of-3d-human-mini-brain-to-improve-glioblastoma-treatment.html).

### Example 4

The objective of this example is to disclose a strategy to minimize the number of samples required to quantify paired and higher order interactions, namely, multiple-body interactions.

In terms of compound-compound interactions, conventional pharmacological and toxicological studies focus only on paired interactions. Chou (2006) presented a general equation (Equation 1) equating cellular responses to the fraction of cells affected fₐ, and dosage of a corresponding compound i, Dᵢ:

$$R = {( \frac{{f}_{a}}{{f}_{u}} )}^{1 {/}_{m}} = {\sum }_{i = 1}^{N} {β}_{i} {D}_{i} + {\sum }_{i > j}^{N} {β}_{i j} ( r ) {D}_{i}^{μ} {D}_{j}^{v} = {β}_{t} {D}_{t} \qquad ( 1 )$$

where fᵤ=1−fₐ is fraction of cells unaffected, βᵢ is activity of compound i (Chou (2006) showed that βᵢ is the inverse of EC₅₀, concentration produces maximum 50% of response), total concentration of the mixture is Dₜ=Σᵢ₌₁^{N}Dᵢ and βₜ is the activity of the mixture. Here a more general interaction coefficient is defined as βᵢⱼ(r)=\hat{β}ᵢⱼr^{x} and r is the ratio Dⱼ/Dᵢ instead of βᵢβⱼ defined by Chou (2006). Furthermore, the undetermined superscripts μ, ν and x are analogous to Hill coefficients in the Hill's equation representing the degree of interactions between compounds. Here, multiple body interaction is neglected because it is generally assumed that this type of interactions is unimportant, and the complexity involved is basically unmanageable. A recent publication indicated that multiple body interactions are important in a biological system and cannot be ignored (Tekin, White et al. 2018). However, as the number of interacting species increases, the number of studies required to accurately quantify these interactions are in the upwards of billions (FIG. 1). It is apparent that when it comes to mining the actives and contributing components of TCM formulas, multiple body interactions cannot be ignored, and a strategy for simplifying the mining process is required.

An approach is being tested: Cluster expansion. Following the assumption that higher order interactions can be expanded in terms of single compound effects and paired interactions, the theoretical frameworks have been developed and experimental data are required for verifications.

### Example 5

Currently, there are no methods available to accurately estimate hepatic clearance and intrinsic clearance in human without running a human study. One of the objectives of this example is to disclose a method to better estimate hepatic clearance of a compound in vivo using data produced from a 3-D hepatic model and a novel method to estimate in vivo metabolism using existing human data of a known set of substrates. TWO approaches are used to estimate in vivo hepatic clearance in human.

The first approach involves: 1. Establishment of a 3-D liver model; 2. Selection of a cocktail of substrates with known human hepatic clearance values and extraction ratios ranging from low (˜0.1), medium (˜0.5) to high (˜1.0), for example, antipyrine, E=0.1, midazolam, E=0.5 and propranolol, E=0.99. These substrates will be used as quality control of the model. Hepatic extraction ratio (E) in vivo is calculated using the following equation:

$$E = \frac{{Cl}_{H}}{Q} \qquad ( 2 )$$

Where, Cl_{H} is hepatic clearance and Q is hepatic blood flow, set at 1.0 L/min.

The metabolic rate of compounds in a fraction will be measured using the 3-D model. Their intrinsic clearance (Vₘₐₓ/Kₘ) values will be estimated for individual compounds. In vitro extraction ratio will be estimated using the following equation:

$$E = \frac{{f}_{u} * {Cl}_{int}}{Q + {f}_{u} * {Cl}_{int}} \qquad ( 3 )$$

wherein, fᵤ is free fraction, Clᵢₙₜ is intrinsic clearance and Q is hepatic blood flow rate (1.0 L/min).

Scaling factor (SF) for Clᵢₙₜ from in vitro to in vivo is calculated using equation 4:

$$S F = \frac{{Cl}_{int , vivo}}{{Cl}_{int , vitro}} \qquad ( 4 )$$

SF for each model substrate will have a set of SF values calculated.

In vivo E values can be estimated using equation 3.

Another objective of this example is to engage the 3-D hepatocyte model to identify MTII compounds in an herbal formula.

Following the guidance of USFDA (https://www.fda.gov/media/82734/download), cocktails of Phase I, Phase II, and transporter substrates will be used as targets for the evaluations (Steps 2-5, FIG. 3). A change in the rate of disappearance of these substrates will indicate the presence of potential inducers or inhibitors.

### Example 6

Cannabis sativa consists of over 554 compounds, of which 113 are cannabinoids, 120 are terpenes (Calvi, Pentimalli et al. 2018), others include amides, flavonoids, phenols, alkaloids, and fatty acids. The quantity of these constituents varies among strains. There exist specialty cultivars which are bred for certain constituents.

It is well-known that the two major cannabinoids: Δ⁹-tetrahydrocannabinol (Δ⁹-THC), psychoactive, is the major component for recreational consumption, and cannabidiol (CBD) non-psychoactive, has numerous medicinal values for conditions such as pain, neurodegeneration, digestive disorders, etc. (Walter and Stella 2004, Nagarkatti, Pandey et al. 2009, Urits, Borchart et al. 2019).

When given in pure forms, THC and CBD are not as effective as that in cannabis. This entourage effect has led researchers to come to the conclusion that other constituents in cannabis could either be active or has the ability to enhance the effects of the major constituents or act synergistically with each other (Russo 2018).

Since the number of constituents is high (>500), the permutations of cross-reactivity among them are daunting, rendering them impossible to quantify. Initial estimates suggest that billions of five-body (five compounds interacting with each other) interactions are present for five hundred compounds, a much higher number of studies is necessary to have an initial understanding of entourage effects, not a good way to develop a medical cannabis product.

The objective of this example is to examine the likelihood of a constituent which may have significant contributions to the overall effects of Cannabis sativa.

The parameter used for the evaluation is maximum exposure index (MEI), a product of AUC and the highest percentage recorded for Cannabis. MEI values of THC and CBD are used as reference constituents because these two compounds are highly active, abundant, and most studied.

Table 1 summarizes the abundance of seven most studied cannabinoids and 14 terpenes reported in the literature (Tubaro, Giangaspero et al. 2010). Twenty-one compounds (Table 1), account for a minimum of 65%, the rest of the 500 plus constituents account for a maximum of 35%, of the plant.

When the systemic exposure (area under the plasma concentration-time curve (AUCₚₗₐₛₘₐ)) and abundance are considered (MEI), the importance of a constituent could change significantly. For example, systemic exposure, in terms of AUC values, is six times higher for CBD when compared to THC, but, when abundance is considered, THC could account for a bigger share of the activity when an extract of Cannabis, rich in THC, is given (Table 2).

CBN is a decomposition product of THC; its accumulation in the bud increases when cannabis flowers are left to cure.

Among the seven cannabinoids listed on Tables 1 and 2, THC, CBD, THCV and CBG have the highest MEI values. Among the 17 terpenes, myrcene, β-caryophyllene, α-pinene, β-pinene, terpinolene, trans-ocimene and limonene, in terms of MEI values, could have significant contributions to the overall efficacy of Cannabis sativa.

TABLE 1
Relative abundance of the most recorded constituents of Cannabis

| | Lower Range, % | Upper Range, % |
|---|---|---|
| Compound Name | total mass | total mass |
| Cannabinoids | | |
| Δ⁹-tetrahydrocannabinol | 0.1 | 25 |
| (THC) | | |
| Δ⁹-tetrahydrocannabivarin | 0.0 | 1.36 |
| (THCV) | | |
| Δ⁸-tetrahydrocannabinol | 0.0 | 0.1 |
| (Δ⁸-THC) | | |
| Cannabidiol (CBD) | 0.1 | 2.89 |
| Cannabichromene (CBC) | 0.0 | 0.65 |
| Cannabigerol (CBG) | 0.03 | 1.15 |
| Cannabinol (CBN) | trace | 1.00** |
| Terpenes | | |
| Myrcene | 29.4 | 65.80 |
| β-caryophyllene | 19.5 | 31.40 |
| α-pinene | 6.0 | 8.40 |
| β-pinene | 1.3 | 1.6 |
| Trans-ocimene | 2.3 | 5.7 |
| Cis-ocimene | 0.2 | 0.3 |
| Limonene | 0.9 | 1.5 |
| Linalool | 0.002 | 0.002 |
| β-phellandrene | 0.4 | 0.4 |
| β-selinene | 0.008 | 0.008 |
| Terpinolene | 3.4 | 5.6 |
| Borneol | 0.008 | 0.008 |
| Cis-β-farnesene | 0.6 | 0.9 |
| α-trans-bergamotene | 0.4 | 0.6 |
| Total | 64.65 | 153.37 |

* not recorded
**https://www.leafly.com/news/cannabis-101/what-is-cbn-and-what-are-the-benefits-of-this-cannabinoid

TABLE 2
Reported constituents of Cannabis sativa with potential clinical efficacy

| | Pharmacokinetic Properties* | | | | | MEI, |
|---|---|---|---|---|---|---|
| | | t_{1/2}, | V_{d}, | Cl_{TB}, | AUC, | AUC* % |
| Compound Name | F, % | h | L/kg | ml/min | ng*h/ml** | mass |
| Cannabinoids | | | | | | |
| Δ⁹- | 16.70 | 1.07 | 1.56 | 1180 | 2.36 | 59.00 |
| tetrahydrocannabinol | | | | | | |
| (THC) | | | | | | |
| Δ⁹- | 46.60 | 1.60 | 1.46 | 742 | 10.47 | 14.24 |
| tetrahydrocannabivarin | | | | | | |
| (THCV) | | | | | | |
| Δ⁸- | 17.90 | 1.10 | 1.56 | 1147 | 2.60 | 0.26 |
| tetrahydrocannabinol | | | | | | |
| (Δ⁸-THC) | | | | | | |
| Cannabidiol (CBD) | 48.43 | 1.68 | 1.47 | 709 | 11.38 | 32.89 |
| Cannabichromene (CBC) | 12.16 | 0.64 | 0.95 | 1206 | 1.68 | 1.09 |
| Cannabigerol (CBG) | 25.40 | 0.92 | 1.13 | 996 | 4.25 | 4.89 |
| Cannabinol (CBN) | 2.96 | 1.05 | 1.61 | 1233 | 0.40 | 0.40 |
| Terpenes | | | | | | |
| Myrcene | 46.43 | 1.94 | 2.04 | 848 | 9.13 | 600.75 |
| β-caryophyllene | 12.98 | 2.18 | 2.64 | 979 | 2.21 | 69.39 |
| α-pinene | 40.73 | 2.49 | 2.66 | 776 | 7.83 | 65.77 |
| β-pinene | 40.71 | 3.18 | 3.45 | 878 | 7.73 | 12.37 |
| Trans-ocimene | 44.63 | 1.44 | 1.56 | 878 | 8.47 | 48.28 |
| Cis-ocimene | 44.63 | 1.44 | 1.56 | 878 | 8.47 | 2.54 |
| Limonene | 52.69 | 2.85 | 2.73 | 775 | 11.33 | 17.00 |
| Linalool | 64.10 | 2.06 | 1.56 | 613 | 17.42 | 0.03 |
| β-phellandrene | 52.96 | 2.22 | 2.15 | 735 | 11.29 | 4.52 |
| β-selinene | 8.26 | 2.48 | 3.41 | 1110 | 1.24 | 0.01 |
| Terpinolene | 52.98 | 2.85 | 2.73 | 775 | 11.40 | 63.84 |
| Borneol | 57.82 | 2.13 | 1.98 | 752 | 12.82 | 0.10 |
| Cis-β-farnesene | 2.44 | 1.14 | 1.59 | 1130 | 0.36 | 0.32 |
| α-trans-bergamotene | 3.51 | 1.31 | 2.01 | 1245 | 0.47 | 0.28 |

*Pharmacokinetic parameters are obtained from ADMET and Sinoveda Canada Inc's proprietary physiologically based pharmacokinetic program.
**AUC is calculated based on a 1 mg dose of a constituent.

Consistent with the results presented in this Example, cannabinoids and terpenes with high MEI values are targets for research (https://www.leafly.com/news/cannabis-101/list-major-cannabinoids-cannabis-effects).

Higher exposure to the brain provides a better likelihood of exerting central nervous system (CNS) effects. Despite some variability, all identified cannabinoids have better brain exposure than THC and CBD (ADMET estimations).

The results of this example highlight several important points: 1. Known cannabinoids and terpenes account for a high percentage of the content of Cannabis; 2. These cannabinoids distribute to the brain extensively, indicating central nervous system (CNS) effects could likely be prominent; 3. As reported in the literature, terpenes, with a much higher quantity and MEI value, could alter the activity of 41 cannabinoids; and 4. Cannabinoids and other constituents with favorable drug-like properties, albeit small in quantities, may contribute to the well-known entourage effects reported in the literature.

### Example 7

The objective of this example is to evaluate the pharmacokinetic properties of known cannabinoids in Cannabis sativa.

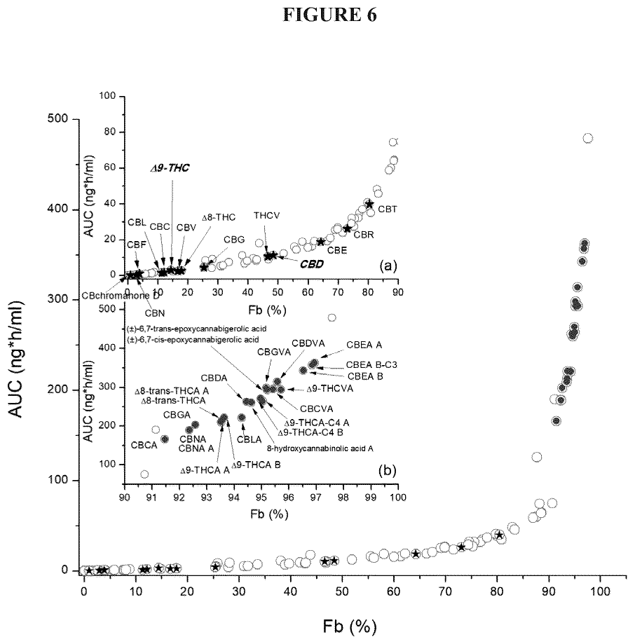

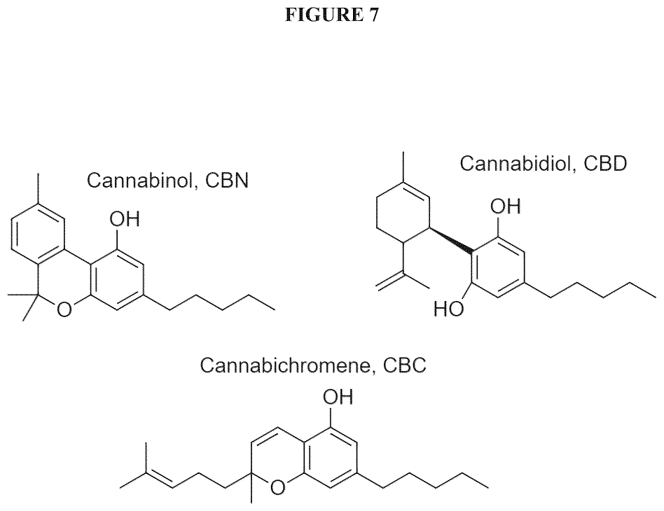

The pharmacokinetic properties of 125 reported cannabinoids were estimated using ADMET. Results are shown in FIG. 6.

Oral bioavailability ranges from 5.4% for cannabinol methylether to 100% for (1'S)-hydroxycannabinol. AUC values normalized to a 1-mg dose range from 0.01 to 479 ng*hr/ml, a span of 48,000-fold.

In terms of systemic exposure, AUC, Δ⁹-THC and CBD ranked 98 and 66, respectively. Both cannabinoids have a bioavailability value less than 50%, THC (16.7%) and CBD (48.4%) (FIG. 6, inset a).

25 cannabinoids have a bioavailability value higher than 90%, 22 of them are in acidic forms (FIG. 6, inset b). These compounds show that their AUCs are 6.6 to 42 times higher than that of CBD and 32 to 203 times higher than that of Δ⁹-THC.

Theoretically, some of these 25 cannabinoids with a high AUC value could be up to forty times more effective than CBD, or up to two hundred times more effective than Δ⁹-THC. Consequently, doses of these cannabinoids could be 5 to 200 times lower than that of CBD or Δ⁹-THC, the two most studied cannabinoids in Cannabis sativa, still but would be equally effective. Cannabinoids with good drug-like properties, like that of the acidic forms, could be more effective if their individual activities are equal to or better than Δ⁹-THC or CBD, or two or more of these compounds act synergistically together.

Pharmacokinetically, Δ⁹-THC and CBD belong to the lower half of the ranking, there is a distinct possibility that more potent cannabinoids are yet to be identified.

In silico estimation of the pharmacokinetic properties of these cannabinoids may not be accurate; however, the rank order of these compounds is consistent with the limited clinical data published in the literature.

The primary objectives of this invention are to use PPT-II to unveil the yet to be discovered cannabinoids, whose effects could overshadow Δ⁹-THC and CBD combined, and their mutual interactions. In the subsequent examples, compositions of cannabinoids are mined using PPT-II for the treatment of hepatocarcinoma (HCC).

### Example 8

The objectives of this example are to: 1. examine potential advantages of using combination approach to derive a formula consisting of three cannabinoids for the treatment of HCC; 2. molecular mechanisms of these combinations; and 3. the importance of ratios of these active ingredients.

Overview: Hepatocellular carcinoma (HCC) is a malignant disease with unfavorable patient outcomes. Less than 35% of those diagnosed with the disease survive 5 years. This number is reduced to less than 12% if the cancer spreads to nearby tissues and less than 2% if the cancer metastases to other organs (Kitisin, Packiam et al. 2011). To date, no approved phytocannabinoid-derived chemotherapeutic treatment option for HCC exists. HepG2 cells, originally isolated from a 15-year-old boy in 1975, is well differentiated and characterized and has long served as a model for hepatocellular carcinoma.

The medicinal use of cannabis dated as far back as 500 BC when gold vessels containing cannabis residue were unearthed in Scythian tombs. Today, cannabis has been approved in the US and Canada as an adjunct to chemotherapy to alleviate nausea and vomiting, loss of appetite, and pain (Kleckner, Kleckner et al. 2019).

The anti-cancer properties of cannabinoids and other constituents in cannabis, such as terpenes, flavonoids, etc., have been reported (Blasco-Benito, Seijo-Vila et al. 2018). In the cannabis arena, Δ⁹-tetrahydrocannabinol (THC) and Cannabidiol (CBD) have been the focus of cancer research for a few years. CBD, due to its lack of central nervous system (CNS) “side effects” has drawn a lot of more interests.

CBD is known to have poor oral bioavailability and drug-like properties (Meyer, Langos et al. 2018). Its low solubility and high first-pass metabolism make CBD a poor candidate for oral delivery. Oral bioavailability of CBD ranges from 9-30%, which is accompanied by huge inter-individual variations in plasma profiles.

Nanoparticles, including liposomes, have been used to enhance the oral bioavailability of CBD. However, there is no parenteral formulations available which are designed to specifically deliver CBD to the liver.

In vivo pharmacokinetic data are not available for Cannabichromene (CBC) and Cannabinol (CBN). In this invention, human pharmacokinetics of CBC and CBN are estimated in silico and will be estimated in vitro. The goals are to determine the optimal routes of administration and the desired dosage forms for delivery.

Entourage effects of cannabis have been well-documented. For example, the effects of CBD are less effective when compared to a cannabis extract containing the same amount of CBD (Blasco-Benito, Seijo-Vila et al. 2018). There are attempts to decipher contributing constituents, but data in the literature are sketchy. Constituents in cannabis have also been reported to work antagonistically together. These two opposing forces make cannabis extracts with undefined chemical profiles questionable when it comes to medicinal use.

In one embodiment, potential synergistic anti-cancer activities of cannabinoid combinations were explored with the aim to identify candidate combinations that are at least as equipotent as sorafenib in the treatment of HCC.

In one embodiment, the mechanisms of action among identified candidates are elucidated.

While numerous studies on cannabis and its constituents support their efficacies against cancer cell proliferation, discrepancies in the literature exist. These could result from differences in experimental design, and/or the underlying genetic and environmental factors characteristic to different cancer types. For example, McKallip, Nagarkatti et al. (2005) demonstrated a lack of responsiveness to pure THC in various breast cancer cell lines, however, Blasco-Benito, Seijo-Vila et al. (2018) reported contradictory results. The latter study further observed an improved response from MDA-MB-231 compared with SUM-159 cells. Interestingly, both of these cell lines are of the overly aggressive triple-negative (ER−, PR−, HER2−) type. In addition to variable cell-line responsiveness, the authors also observed a significant improvement with botanical extracts containing THC, other cannabinoids, flavonoids, and terpenes compared to pure THC alone for all the cell lines tested.

The effects of cannabinoids can be explained by their variable binding affinities to multiple G-coupled protein heterodimeric receptors such as CB1, CB2, GPR55, and TPRV1, conveying multiple downstream pathways resulting in variable drug responsiveness (Moreno, Cavic et al. 2019). It has been reported that THC can directly augment AMPK mediated autophagy of hepatocellular carcinoma (HCC), HepG2 cells, through direct binding of CB2 receptors (Vara, Salazar et al. 2011). This is in addition to THC induced autophagy mediated through ALK receptors in glioma cells (Lorente, Torres et al. 2011). Whether these responses are unique to the various cell lines or ubiquitous among various cancer cell types is yet unresolved. More so, Torres, Lorente et al. (2011) demonstrated a synergistic response to THC with the concurrent treatment of a selective ALK inhibitor, TAE-684, indicating the additive or synergistic effects can be augmented by targeting convergent pathways.

An emerging therapeutic target are the agonistic or antagonistic compounds to modify heterodimer formations within numerous C-coupled protein receptors, which cannabinoid receptors are part of. This opens a plethora of new disease pathways as this superfamily of receptors comprises about 4% of the protein coding genome (Moreno, Cavic et al. 2019). New targets may provide mechanistic description of cannabinoid and/or other compound interactions.

While THC is known to bind the peripheral CB2 receptors in HepG2 cells and induce pro-apoptotic events, CBD binds to TRPV1, PPAR, GPR55 and TRPM8 receptors and can induce apoptotic events from increased reactive oxygen species (ROS) and ceramide. Comparatively, Zhong (2020) reported that CBN mediates apoptosis through the MAPK/ERK and PI3K-ATK pathways and cell cycle arrest by downregulating P21. Interestingly, Zhong's work also demonstrated a downregulation of CB2 and GPR55 receptors from CBN treatment. This would presumably have a direct effect on the efficacy of CBD's activity with concurrent treatment. On the other hand, CBC is a TPRA₁ agonist (De Petrocellis, Vellani et al. 2008) and mediates increased intracellular Ca²⁺, while at the same time inhibits AEA reuptake. These reports demonstrate the multitude of receptor mediated pathways that cannabinoids can influence autophagy and apoptosis for the treatment of cancer. Couple these various receptor preferences with variable affinities, and these agents might work additively, synergistically, or antagonistically within a given cell line. Indeed, Blasco-Benito, Seijo-Vila et al. (2018) demonstrated an improved response with botanical drug extracts compared to pure THC for all the cell lines examined. Given the number of permutations and responses that could result from botanical substances, it is not unreasonable to expect variable responsiveness from cannabinoid extracts that contain any number of these cannabinoids in any number of combinations or relative ratios, not to mention potential additional anti-cancer agents such as terpenes and flavonoid anti-oxidants (Tomko, Whynot et al. 2020). All in all, it would be safe to conclude that treatment with poorly characterized extracts would produce unpredictable responses.

With that in mind, there exist a growing number of studies and patents validating the use of pure mono-cannabinoid compounds such as tetrahydrocannibinolic acid (THCA), cannabidiolic acid (CBDA), cannabigerolic acid (CBGA), cannabinolic acid (CBNA), cannabichromenic acid (CBCA) and their decarboxylated derivatives as anti-cancer agents (Javid, Duncan et al. 2018, Koltai, Poulin et al. 2019). While these reports have confirmed their in-vitro and in-vivo activity against a variety of cancer cell lines, their relatively low efficacy (IC₅₀ values in the μM range) coupled with poor drug-like properties for decarboxylated forms make them questionable drug candidates. Indeed, Millar and colleagues have demonstrated this with CBD (Millar, Stone et al. 2018).

Recent disclosures have reported synergistic interactions with combinations of cannabinoids (Stott, Duncan et al. 2017), however, these studies either have primarily focused on only two cannabinoids with a limited number of defined ratios, or utilize complex trichome extracts containing a multitude of components yet characterizing but a few thereof (Parolaro, Massi et al. 2013). Drawing conclusions from these results are problematic given that any number of ingredients within a complex mixture might be responsible for the observed effect and cross-traffic may exist among convergent biological pathways. It is noted that CBD and CBN both interact with GPR55 receptors. CBD is a GPR55 agonist. CBN has been shown to downregulate GRP55 expression, which could result in a variable response relative to their concentrations and affinity. While CBD and CBC are TRPV1 and TRVA1 agonists leading to increase intracellular calcium and promote apoptosis. CBD also works on different pathways and receptors, some of which may be convergent. On the other hand, because CBC and CBN appear to work on alternate pathways, a more direct competitive/additive/synergistic response may result. With only three cannabinoids, it is easy to see how any number of overall responses may be observed depending on the amounts of each used. It is difficult to predict what the overall effect might be without empirical evidence and what ratios might overload the receptors and shunt the response to other pathways.

Natural fluctuations of cannabinoid ratios within trichomes result from any number of factors including but not limited to cannabis strain, growing conditions, and harvest times. Thus, one can conclude that variable responses toward hepatic carcinoma cell lines would result from different botanical extracts. Furthermore, predicting their anti-cancer effects becomes problematic. The degree of predictability is further compromised by other endogenous phytocompounds, which may promote anti-cancer activity in numerous cell studies. This too has been demonstrated in a various cell studies (Tomko, Whynot et al. 2020).

This disclosure reveals defined combinations of cannabinoids with convergent receptor targets and pathways that are known to induce pro-autophagy and apoptotic events in cancerous cells. The combination dosage form of CBD and the multiple cannabinoid combinations described in this invention present IC₅₀ values comparable to sorafenib against HepG2 cells.

Summary of Combination Candidate Development: In one embodiment, three compositions of cannabinoids, CBD, CBC/CBN and CBD/CBC/CBN which have efficacies comparable to sorafenib, a first line chemotherapeutic agent, in an in-vitro liver cancer model. In another embodiment, the mechanisms of synergism of CBC/CBN and CBD/CBC/CBN are revealed.

This disclosure reveals three compositions of cannabinoids comprising of CBD, CBD/CBC and CBD/CBC/CBN with divergent receptor targets and pathways that are known to induce pro-autophagy and apoptotic events in cancerous cells. The three compositions described in this invention are more effective than sorafenib against HepG2 cells.

The efficacies of these three compositions will be enhanced when they are nano encapsulated. These nanoparticles will be designed specifically for liver delivery to enhance efficacy and safety.

In one embodiment, three compositions of cannabinoids, CBD, CBC/CBN and CBD/CBC/CBN which have efficacies better than sorafenib, the first-line chemotherapeutic agent, in an in vitro liver cancer model.

In another embodiment, the mechanisms of synergism of CBC/CBN and CBD/CBC/CBN are revealed.

In another embodiment, modes of delivery to achieve/provide the best efficacy and safety profiles are disclosed.

In another embodiment, nano-encapsulated dosage forms for CBD, CBC/CBN and CBD/CBC/CBN for parenteral administrations will be revealed.

In another embodiment, nano-encapsulated forms of the composition will be shown to be more effective than their respective non-capsulated compositions.

The dosage of the nano-encapsulated dosage forms will be at least three times less than their non-capsulated counterparts.

Detailed Description of Combination Cannabinoid Formulas Development: In this invention, it is disclosed that compositions comprising of CBD, CBC/CBN and CBD/CBC/CBN are more potent compared to the liver chemotherapeutic agents, sorafenib. An in-vitro cell model, HepG2, quantitation methods, HPLC/MS/MS, gene expression studies, Western-blot studies, and data analysis for compound-compound interactions were used to evaluate relative efficacy and mechanisms of interactions.

Materials and Methods: Pure cannabinoids were obtained from Supelco/Cerilliant. Cannabidiol (CBD, C-045-1ML, lot FE10071912), cannabichromene (CBC, C-143-1ML, lot FE06152005), cannabinol (CBN, C-046-1ML, lot Positive FE05052008). control compounds, regorafenib (TCI, R0142-25 MG, lot 11-88997-23096), and sorafenib (Selleckchem, S7397, lot S739707) were obtained from Fisher. Control chemicals were reconstituted in DMSO to 10 mM and subsequently diluted to working concentrations. Pure cannabinoids in methanol (1 mg/ml) were added directly to media at the highest concentration assessed and serially diluted to subsequent working concentrations.

Cell assays: HepG2 cells were obtained from the American Type Culture Collection (ATCC). Cryopreserved cells in DMEM with 10% DMSO were thawed and diluted 10-fold in phenol red free DMEM media containing fetal 10% bovine serum, 100 μg/ml penicillin/streptomycin, supplemented with Glutamax™ and sodium pyruvate. Subsequently, cells were centrifuged at 20 g for 10 minutes. The cells were resuspended in the same media to a concentration of 2×10⁵ cells/ml. Ninety-six well cell culture treated flat bottom plates were seeded with 7.5×10³ HepG2 cells and incubated at 37° C., 5% CO₂, >95% relative humidity for 18 hours to allow cellular adhesion after which media was replaced with like media containing 1-20% FBS and various test compounds (50 μg/ml to 50 ng/ml). This media with test compounds was replenished after 24 hours. After 48 hours (2 doublings of cells), sodium; 2-(2-methoxy-4-nitro-5-sulfonatophenyl)-3-(2-methoxy-4-nitro-5-sulfophenyl)-N-phenyltetrazol-3-ium-5-carboximidate (XTT) (0.3 mg/ml) was added, and cells incubated for 2 hours. Formazan production was measured 450 nm Varioskan lux at on a spectrophotometer. Non-specific absorption was measured at 660 nm and subtracted from the reference measurement at 450 nm. Sample measurements were obtained in triplicates (n=3). Following blank subtraction, the data were averaged and fit to four-point logistic regression curves to obtain IC₅₀ values (R²>0.95). Blank samples included ethanol as a negative control, which was the solvent for the commercially acquired cannabinoids. The ethanol concentrations for blank standards reflected those used in successive cannabinoid serial dilutions. Initial studies were performed to assess single cannabinoid activity toward HepG2 cell proliferation.

Ternary cannabinoid assays: For tertiary cannabinoid compound mixtures, ratio mixtures of CBD:CBC:CBN were randomly chosen between 9:3:1 to 1:1:1 and CBN:CBC:CBD between 3:2:1 to 1:1:1 in IMDM media supplemented with 20% FBS and 1% ethanol and CBD:CBC:CBN ratios between 9:3:1 to 1:1:1 in DMEM media supplemented with 1% FBS. Cannabinoids were mixed and diluted in the media ready for serial dilution. Cell assays and XTT experiments were conducted as described above. XTT values were plotted relative to the first two components ratio to assess ideal responses relative to the primary component.

Spheroid (solid tumor) assay: HepG2 cells were thawed and reconstituted in Williams E media supplemented with Gibco hepatocellular maintenance supplement and 1% FBS. Cells were seeded into Corning® spheroid black-walled, clear round-bottomed, ultra-low attachment, 96-well microplates at a density of 1500 cells/well. The plates were spun at 10×G for 20 minutes to congregate cells into the center of the wells. After a 60 hour incubation at 37° C., 5% CO₂, spheroids were subsequently treated with the cannabinoid mixture CBD:CBC:CBN 9:3:1 and incubated a further 48 hours. Spheroid vitality was measured with Invirogen's CyQuant™ proliferation assay (excitation/emission: 508/527 nm) following 60 minutes at 37° C.

Mass Spectrometry: HPLC-DAD/MS techniques were used to confirm concentrations and ratios of individual cannabinoids. Random samples (50 μl) were taken from the test set after 30 minutes of incubation and crashed with 150 μl of methanol to precipitate media proteins. The samples were centrifuged at 21910 RCF for 5 minutes and the supernatants were collected into 2 mL glass vial with insert. Quantitative data were acquired using an Agilent 6410 triple quadrupole mass spectrometer coupled to a 1260 series HPLC and UV detector. A thermostatic autosampler at 4° C. was used for injecting 5 μL of the samples into a Phenomenex Kinetex 5 μm XB-C18 100 Å, 250×4.6 mm column equipped with a C18 SecurityGuard ULTRA Cartridge at 40° C. with a flow rate of 1.00 mL/min and a gradient of 72 to 100% methanol in 45 minutes. The mobile phase A contained water with 0.05% ammonium acetate and mobile phase B consisted of methanol with 0.05% ammonium acetate. UV detection was used at 215 nm with a signal bandwidth of 4 nm. Selected ion monitoring (SIM) LC/MS analysis was performed with the following parameters at negative ion mode: The source gas temperature was set at 320° C. with a flow rate of 12 L/min, the capillary voltage maintained at −3.8 kV, dwell times were set at 200 ms with the fragment voltage set at 135 V and gas nebulizer pressure was set to 35.0 psi. Concentrations were determined by quantifying the area response of the samples against a standard calibration curve with a range from 0,025 to 10 μg/ml for the MS detector and a range from 0.5 to 15 μg/mL for the UV detector. All the certified reference materials were purchased from Cerilliant Corporation (CBD, C-045; CBC, C-143; CBN, C-046).

rt-PCR: Target cancer gene pathways will be analyzed using a custom Taqman array card exploring 91 unique genes involved with a few cancer pathways.

Two million HepG2 cells in IMDM media with 20% FBS, and 1% ethanol were seeded in T75 flasks and incubated at 37° C., 7.5% CO₂ until 70-80% confluent (approx. 7.5×10⁵ cells). The media were replenished with media containing individual cannabinoids (CBD 17 μg/ml, CBC 23 μg/ml, CBN 20 μg/ml) and cannabinoid mixture of CBD:CBC:CBN of 1:1:1 (17 μg/ml) and incubated for 6 hours prior to harvest. Sorafenib (12 μg/ml), a kinase inhibitor with anti-angiogenic and anti-proliferation activity mediated through Raf, VEGFR, and PDGFR (Roberts and Der 2007) was included as a positive control. Untreated cells were included as a negative control Glyceraldehyde-3-phosphate dehydrogenase (GAPDH) was included as an endogenous control and used for normalization of the data.

RNA was extracted using Invitrogen's Dynabead mRNA DIRECT kit using the recommended protocol to provide 50 μL of a purified mRNA solution. Fifty L of RT master mix was mixed into the mRNA solution and the mixture left to react at 37° C. for 60 minutes. Reverse transcriptase inactivation was performed at 95° C. for 5 minutes and the resulting cDNA samples were kept at 4° C. until use. Fifty-five microliters of TaqMan Fast Advance master mix were added to an equal amount of cDNA sample containing 1 μg/100 μl of cDNA, which was loaded into each card reservoir. The cards were centrifuged twice at 1200 RPM for 3 minutes in a Legend XFR centrifuge and the rtPCR reaction was conducted in a QuantStudio7 PCR instrument using the standard settings with 40 amplification cycles.

In-cell Western-blot analysis: Protein expression was assessed by seeding 1000 HepG2 cells/well from cyro frozen stocks into 384 well optical bottom black culture plates from Thermo-Scientific (item 142761) in IMDM media with 20% FBS and 1% ethanol. After a 48-hour recovery, the media was replaced with the same media containing (CBD: 17 μg/ml; CBC 23 μg/ml; CBN 20 μg/ml; Sorafenib 12 μg/ml; SCI-111 17 μg/ml; SCI-521:17 μg/ml). After a six hour treatment, the cells were fixated with 4% formaldehyde for 15 minutes, permeated with 0.1% triton-x100 for 10 minutes, and left in blocking buffer (phosphate buffered saline (PBS) with 3% bovine serum albumin (BSA)) overnight at 4 degrees Celsius. The following day, the cells were rinsed with PBS, incubated with 25 μl PBS containing primary antibody (Table 1) for 3 hours at 25° C. Subsequently, the primary antibody was removed, cells were rinsed twice with PBS, and incubated with 25 μl of PBS containing the secondary antibody (Rabbit: Alexa Fluor 790 donkey anti-rabbit IgG (H+L) Lot 2409042; Mouse: Alexa Fluor 790 donkey anti-mouse IgG (H+L) Lot 2300923) at a concentration of 1:2000. Draq5 (1:2000) was added for normalization of data. After an hour incubation in the secondary antibody, the cells were rinsed with PBS and readings at 700 and 800 nm were acquired on a Licor Odyssey fluorescence imager with a resolution of 21 μm. Analysis was subsequently performed with Empiria Studio 2.1. A total of 5 samples were collected per antibody and the sample size was reduced to 3 for the statistical analysis.

TABLE 3
List of primary antibodies selected for Western-Blot analysis

| Primary | | | |
|---|---|---|---|
| Antibody | SKU | Secondary | Ratio |
| ERBB2 (HER2) | MA514509 | Rabbit | 1 to 250 |
| VEGA | 22341-1-AP | Rabbit | 1 to 50 |
| P53 | MA515244 | Mouse | 1 to 100 |
| C-Met | PA532357 | Rabbit | 1 to 100 |
| C-Met-PO4 | 710792 | Rabbit | 1 to 250 |
| MEK2 | PA529291 | Rabbit | 1 to 500 |
| MEK2-PO4 | 44454G | Rabbit | 1 to 250 |
| PIK3CA | MA517149 | Mouse | 1 to 200 |
| PTEN | MA512278 | Mouse | 1 to 50 |
| PTEN-PO4 | 441066G | Rabbit | 1 to 250 |
| C-MYC | 132500 | Rabbit | 1 to 100 |
| DVL2 | PA529244 | Rabbit | 1 to 100 |
| TEAD2 | PA566833 | Rabbit | 1 to 100 |
| CUL2 | 700179 | Rabbit | 1 to 250 |

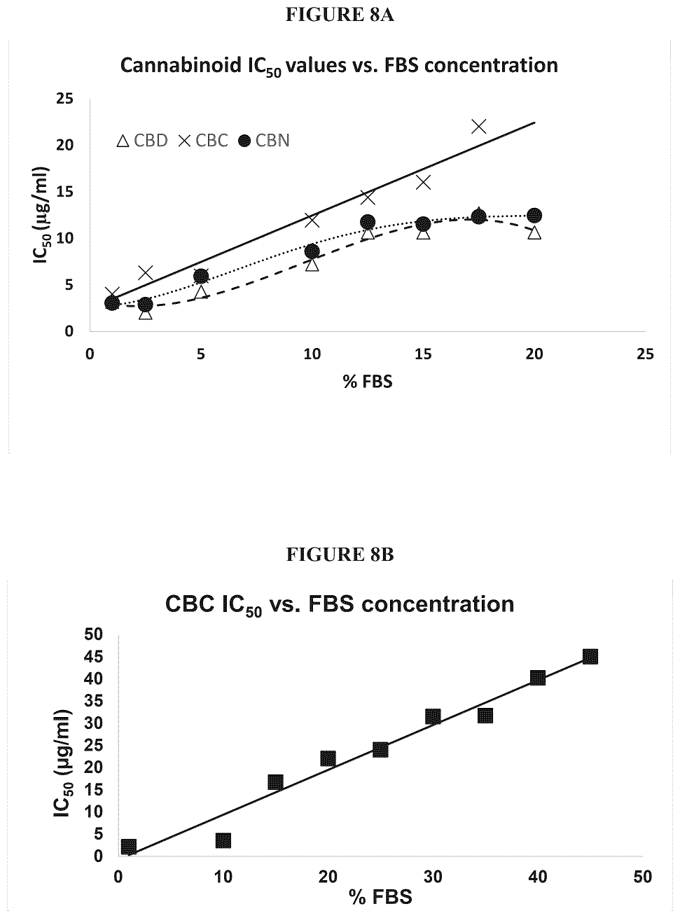

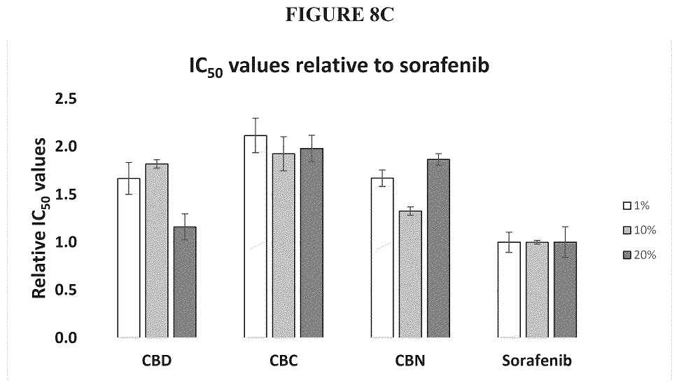

Data collection: Subsequent experiments revealed in this disclosure focused around the three non-psychoactive cannabinoids that presented the lowest IC₅₀ values against HepG2 cells, namely CBD, CBC and CBN. Further investigation of these cannabinoids in various media identified a correlation between FBS concentration in the media and the observed IC₅₀ values (FIG. 8A). Sorafenib, CBD and CBN display an IC₅₀ value leveling, whereby the IC₅₀ values plateau after 12.5% FBS, however, CBC presents a linear correlation that was further measured up to 45% FBS (FIG. 8B). This observation led the inventors to investigate the IC₅₀ values for the individual cannabinoids relative to sorafenib at 1, 10 and 20% FBS. These data are shown in FIG. 8C. As seen, despite the various IC₅₀ values obtained, the cannabinoid IC₅₀ values relative to sorafenib remains at ˜1.25 for CBD, ˜1.5 for CBN, and ˜2.0 for CBC. This methodology allows for a consistent comparison of data across various experimental conditions so long as sorafenib is used as a benchmark standard. Multiple experimental conditions were explored and revealed toxicity from the standard solvent systems reported in the literature (data not shown). As such, IMDM media with 20% FBS and 1% ethanol was found to be a non-toxic system that allowed for a clear interpretation of data of HepG2 cells as was used for the remainder of this disclosure.

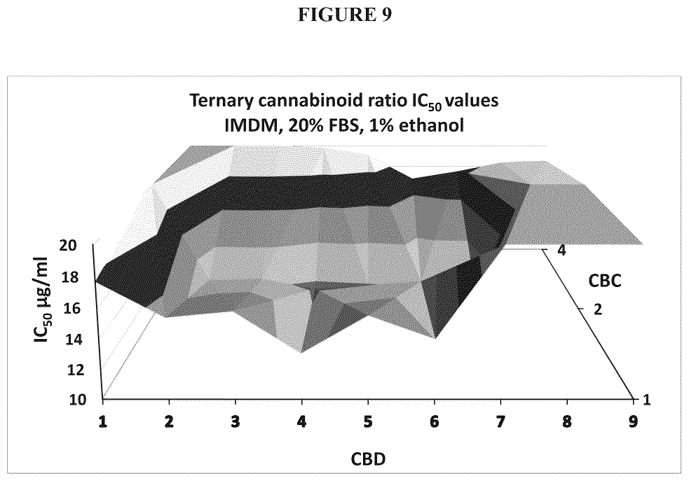

Pairwise Studies: Given the results of pure cannabinoids, the inventors conducted a similar biological study using three cannabinoid pairs, CBD-CBC, CBD-CBN, CBC-CBN with a range of ratios to estimate the effects of a three-component interaction matrix. The resulting IC₅₀ values of the corresponding triple ratio concentrations, are shown in FIG. 9. Optimal effects are observed when the concentration ratios of CBD:CBC exceeded 2:1 and when CBD: CBN were between 2:1 and 4:1 in IMDM media with 20% FBS.

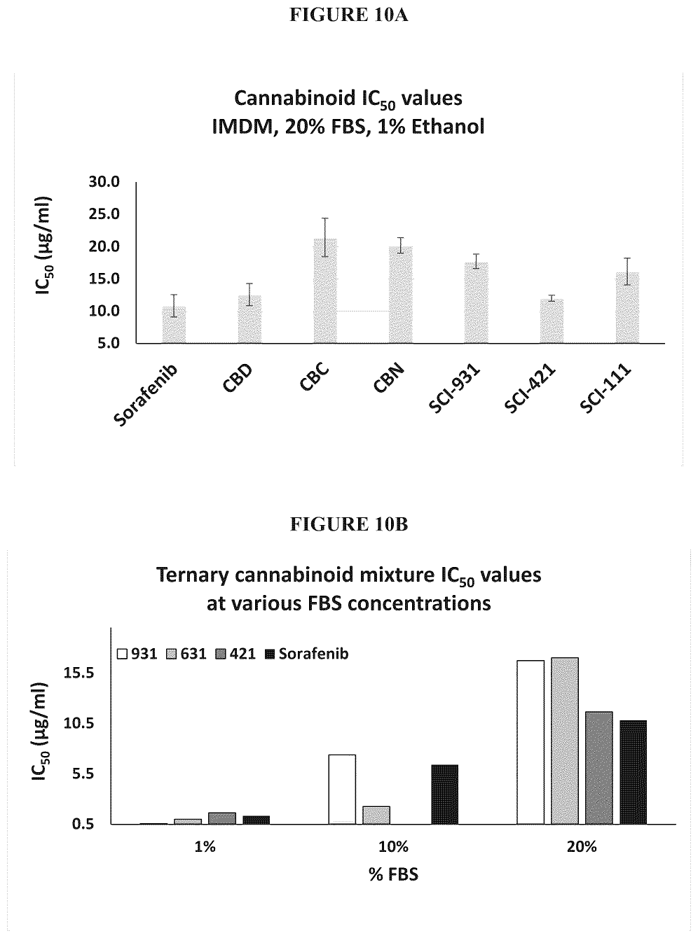

Ternary Interactions: Validation of the XTT results seen in Example 8 were performed to assess potential synergistic interactions of the cannabinoids, CBD, CBC, and CBN. Based on the observed potential synergism ranges from the plots shown in FIG. 9, various combinations for the three cannabinoids were selected for the ternary analysis (CBD:CBC:CBN 9:3:1, 4:2:1, and 1:1:1). IC₅₀ values were obtained using the XTT assay described in this disclosure, and values are reported respective to the total concentration of the combination of cannabinoids used for each sample. Results from this study are shown in FIGS. 10A and 10B. While a CBD:CBC:CBN ratio of 4:2:1 produced an IC₅₀ value comparable to sorafenib in media with 20% FBS, this ratio shifted to 9:3:1 as the FBS concentration was reduced to 1% (FIG. 10B). This shift is hypothesized to result from decreased plasma protein binding of CBC, which resulted in a concurrent reduction in IC₅₀ values (FIG. 8A). Indeed, an optimal IC₅₀ is seen when the relative 3:1 ratio for CBD:CBC and CBC:CBN at 1% FBS requires an increase ratio to 2:1 at 20% FBS, which likely provides a similar free drug concentration of CBC. This set of results provide evidence that combination drug candidates are better than individual compounds, and they are more efficacious than sorafenib. The results also showed that the choice of in vitro model, testing methods and the condition under which the test compounds are being evaluated are of utmost importance. This is especially true when combination drug candidates are being screened.

Mechanisms of Interactions: The objective of this study is the understand the mechanism of actions of the cannabinoids explored in this disclosure and the optimal effective ratio of these compounds against the proliferation of HepG2 cells in-vitro.

Assessment of cancer gene pathways were carried out using custom designed TaqMan array cards exploring 86 genes linked to numerous cancer related pathways. These pathways include top level G-coupled protein receptors (CB1, CB2, GPR55, TRPV1, etc.) and downstream proteins and enzymes linked to apoptosis, autophagy, cell cycle arrest, and including the pathways perturbed by cannabinoids discussed previously in this disclosure. These data are shown in Table 4. Comparison of the cannabinoids against the negative control yielded results consistent with previous reports in the literature. Indeed, this disclosure confirmed that CBD affects the mTor pathway through alterations in several MAP kinase and PI3K pathway genes. We also confirmed CBN likely alters mTOR through the PI3K/ATK pathway and CBC induced CAMK1 and ELK1 genes that are linked to apoptosis. However, our comprehensive analysis reveals a far more extensive involvement of gene expression. Several additional pathways including TGF signaling, Hedgehog, Jak-STAT, Hippo and Notch are perturbed that have not been reported. Interestingly, Hedgehog is not affected by CBD, while Jak-STAT and Notch are only affected by CBC. TGF signaling and PIK3R5 appear to be influenced by CBN to a greater extent. These unique targets might account for the additive effects observed from the combinatorial formulation. Indeed, the CBD:CBC:CBN ratio of 1:1:1, despite only retaining ⅓ of the initial concentration of each individual cannabinoid, while not presenting the best overall IC₅₀, indeed presents with some of the strongest gene fold changes (MAP3K, SMAD3, SMO, CCNE1, and BCL2). These effects suggest up stream interactions may exist which are not identified in this study that ultimately impart synergism by the mixture over any individual cannabinoid alone. In addition to individual pathways, genes linked to specific cellular processes were also explored. As anticipated, genes involved with cell cycle arrest and apoptosis were altered with all three cannabinoids tested. However, genes involved with angiogenesis (VEGFA and CUL2), and cellular adhesion (GSK3, DVLD2, and catenin B) were also altered. These processes are important for metastasis and cannot be assessed from a simple 2D cellular assay and would not influence IC₅₀ values measured from an XTT assay.

In addition to the aforementioned genes, the inventors took note that neither CB1 nor CB2 genes are expressed in the HepG2 cell line studied. Rather PPARG, ERBB2 and MET proteins show change. It is not clear if the cannabinoids bind directly to these receptors or affect their expression. PPARG is a transcription factor linked to the expression/activity of G-coupled protein receptors, and it might affect potential cannabinoid receptors (GRP55 and GRP119). MET is also known as hepatocyte growth factor and is directly linked to hepatocyte proliferation.

TABLE 4
rtPCR of genes presenting Log2 fold changes relative to untreated cells. Highlighted
genes identify candidates selected for confirmatory western blot analysis.

| | | Gene | CBD | CBC | CBN | SCI-111 | Sorafenib |
|---|---|---|---|---|---|---|---|
| Receptors | | PPARG | | −1.826 | z | 2.204 | 2.295 |
| | | | −6.09 | −2.51 | | 1.465 | −1.977 |
| | | | −2.96 | −4.473 | −1.016 | −1.575 | |
| Pathways | MAP kinase | MAP1LC3A | −1.527 | −2.006 | | | 1.819 |
| | | MAP2K1 | | −2.534 | | | |
| | | MAP3K | −1.213 | | 1.518 | 2.993 | |
| | | MAPK8 | −2.27 | −2.068 | −1.284 | | 1.036 |
| | | RAF1 | | | | | |
| | | PAK3 | −1.926 | | | | |
| | | RASA1 | −1.356 | −1.307 | | | |
| | | RAC1 | −1.274 | −1.422 | | | 1.049 |
| | | RASSF1 | | −1.813 | | | |
| | PI3K/AKT/MTOR | AKT1 | | −1.915 | | | 2.71 |
| | | PIK3C2A | −1.161 | −1.77 | −1.181 | | |
| | | | −1.565 | −2.204 | | | 1.737 |
| | | PIK3R5 | | −1.821 | −2.689 | −1.35 | −1.716 |
| | | | −1.174 | −2.291 | | | |
| | | MTOR | −1.055 | −1.847 | | | 1.263 |
| | TGF signaling | SMAD2 | −1.437 | −1.617 | | | |
| | | SMAD3 | | −2.487 | −3.711 | 4.903 | |
| | Ca signaling | CAMK1 | −1.329 | −2.15 | | | |
| | | ELK1 | | −3.143 | −1.026 | | 1.23 |
| | Hedgehog | SMO | | | 2.336 | 3.774 | −3.747 |
| | | SUFU | | −1.668 | | 1.467 | 1.555 |
| | JAK-STAT | STAT1 | | −2.228 | | 1.018 | |
| | | STAT5A | | −1.559 | | | |
| | HIPPO | TAZ | −1.766 | −1.874 | −1.143 | | 1.389 |
| | | | | −1.568 | | | |
| | NOTCH | NOTCH1 | | −1.949 | −1.084 | | |
| Processes | Cell Cycle | CCND1 | −1.396 | −1.206 | | | |
| | Arrest | CCND2 | −1.032 | | | | |
| | | CCNE1 | | | | 2.398 | −2.043 |
| | | CDK1 | −1.257 | −1.725 | | | |
| | | CDK2 | | −1.316 | | 1.417 | |
| | | CDK4 | −1.517 | −1.914 | | | |
| | | CDK6 | −1.224 | −1.735 | | | |
| | | | −1.332 | −2.312 | | | 1.407 |
| | | MNT | −1.533 | −2.804 | | | 2.353 |
| | | PAK3 (P21) | | −1.643 | | | |
| | Angiogenesis | | | −1.521 | | 2.702 | |
| | | | −4.218 | −3.902 | −3.519 | −2.5 | |
| | Apoptosis | APFAF1 | | −1.945 | | | |
| | | BAD | −1.49 | −2.917 | −1.041 | | |
| | | BAX | −1.103 | −2.332 | | | 2.239 |
| | | BCL2 | 4.139 | 3.95 | | 4.684 | |
| | | BCL2L1 | −1.414 | −1.241 | | | 1.716 |
| | | BECN1 | | | | | |
| | | CASP3 | −1.165 | | | | |
| | | | −1.09 | | | | |
| | Cellular | CRK - p38 | | −1.414 | | | |
| | Adhesion | CTNNB1 | −1.164 | −1.487 | | | 1.085 |
| | | | −1.436 | −1.865 | | | 1.497 |
| | | GSK3B | −1.409 | −1.728 | | | 1.136 |
| | | ROCK1 | −1.013 | −1.213 | | | |
| | Drug Resistance | MITF | −4.196 | −4.421 | | | −1.689 |

Genetic Analysis: To validate the PCR observations, candidate genes were selected from Table 4 for protein expression. These genes are highlighted in Table 4. It is common that observed gene expression changes do not always correlate to protein expression due to recycle/scavenger pathways, lag times between gene and protein expression and a complex network of homeostatic mechanisms at play. Regardless, protein expressions of all target genes selected validated PCR results for at least one of the drug candidates screened. These fold changes are shown in Table 5. Most notably, are the ubiquitous changes observed for apoptotic related proteins p53 and c-MYC. Again, cellular adhesion proteins were also influenced by all candidates examined and the angiogenesis related proteins were influenced by CBD and the cannabinoid combinations. The MAPK/PI3K/ATK proteins were also affected by the cannabinoids confirming previous literature findings. Interestingly, the combination formulations tested presented not only with comparable IC₅₀ values in XTT experiments despite a reduction of individual doses of cannabinoids, but also with gene and protein expression changes either similar or more significant relative to their individual component counterparts. For example, SCI-521 contains only 62.5%, 25%, and 12.5% of the individual cannabinoids CBD, CBC, and CBN yet produces a similar IC₅₀ to that of CBD and sorafenib alone in IMDM media with 20% FBS. This combination also produced similar reduction in phosphorylated PTEN protein as CBD alone (Table 5). It also produced more VEGA expression compared to CBD alone and a reduction in CUL2, which was not observed for CBD for the given time frame (6 hours) when samples were taken. These data suggest a co-operative effect of the individual cannabinoids in providing an additive/synergistic effect. Comparable results are observed for SCI-111, which only has 33% of each cannabinoid. While this formulation did not exhibit an ideal IC₅₀ in XTT results in either 1% or 20% FBS, it did produce a more significant protein change for p53 compared to the individual cannabinoids. Based on the data shown in 10B, it can be hypothesized that this ratio would present optimal results when FBS approaches 50%.

TABLE 5
Protein fold changes for candidate genes obtained from in-cell western
blots. SCI-111: CBD:CBC:CBN 1:1:1; SCI-521: CBD:CBC:CBN 5:2:1.

| | | CBD | | CBC | | CBN | | SCI-111 | | SCI-521 | | Sorafenib | |
|---|---|---|---|---|---|---|---|---|---|---|---|---|---|
| | | Fold | | Fold | | Fold | | Fold | | Fold | | Fold | |
| | Gene | change | SD | change | SD | change | SD | change | SD | change | SD | change | SD |
| Receptor | HER2 | 1.34 | 0.15 | | | | | | | 0.73 | 0.13 | 1.29 | 0.07 |
| | MET | | | | | | | | | 1.26 | 0.04 | 0.59 | 0.10 |
| | MET PO4 | 1.52 | 0.12 | 1.80 | 0.11 | | | | | | | 0.73 | 0.00 |
| Pathways | MAP | — | | | | | | | | | | | |
| | kinase/PI3K | | | | | | | | | | | | |
| | PTEN | 0.80 | 0.05 | | | | | 0.75 | 0.10 | 0.72 | 0.17 | | |
| | PTEN PO4 | | | 1.40 | 0.12 | | | | | | | 1.26 | 0.25 |
| | MEK | | | 1.22 | 0.08 | | | | | | | | |
| | MEK PO4 | 1.40 | 0.27 | 1.68 | 0.15 | 1.33 | 0.11 | | | | | | |
| | PI3K | 2.05 | 0.19 | | | 1.46 | 0.10 | 0.75 | 0.12 | | | | |
| | HIPPO | | | | | | | | | | | | |
| | signaling | | | | | | | | | | | | |
| | TEAD2 | 1.46 | 0.12 | 1.34 | 0.11 | 1.35 | 0.07 | | | | | 1.54 | 0.09 |
| Processes | Angiogenesis | | | | | | | | | | | | |
| | VEGA | 1.33 | 0.13 | | | | | 1.25 | 0.07 | 1.61 | 0.19 | | |
| | CUL2 | | | | | | | 1.37 | 0.09 | 0.72 | 0.02 | 1.46 | 0.01 |
| | Apoptosis | | | | | | | | | | | | |
| | c-MYC | 1.55 | 0.14 | 1.70 | 0.06 | 1.40 | 0.05 | 1.66 | 0.10 | 1.46 | 0.03 | 1.20 | 0.09 |
| | p53 | 1.28 | 0.02 | 1.26 | 0.03 | 1.22 | 0.03 | 2.91 | 0.31 | | | 1.22 | 0.15 |
| | Cellular | | | | | | | | | | | | |
| | Adhesion | | | | | | | | | | | | |
| | DVLD | 1.67 | 0.03 | 1.34 | 0.13 | 1.66 | 0.10 | 1.46 | 0.17 | 1.25 | 0.14 | 1.74 | 0.17 |

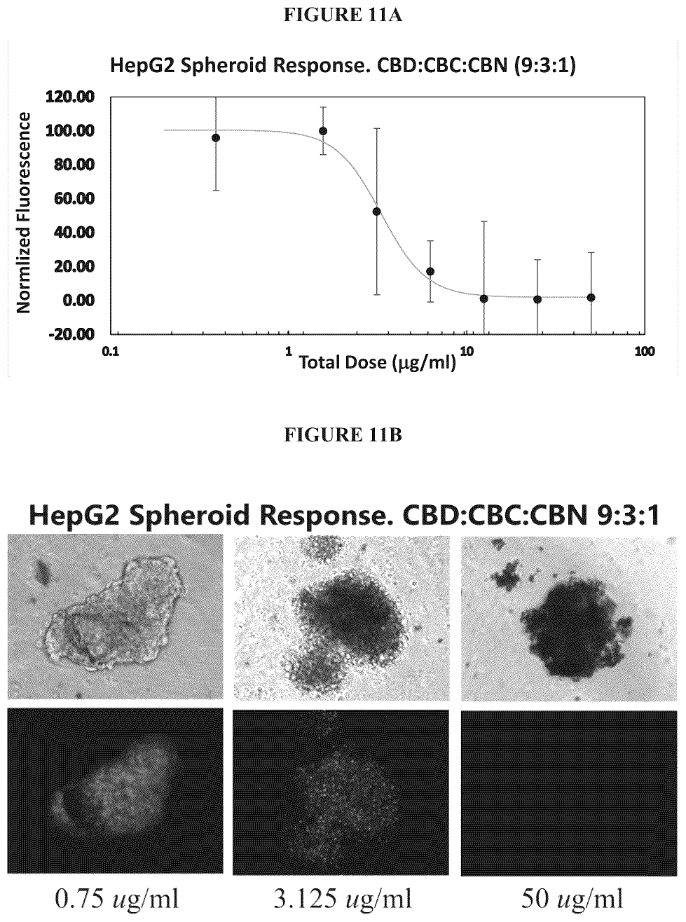

3D HepG2 Study: To compare the efficacy of the cannabinoid mixture toward an in-vitro solid tumor model HepG2 cells were assessed in spheroid form. The cannabinoid mixture (SCI-931: CBD:CBC:CBN 9:3:1) demonstrated activity with an IC₅₀ value of 2.95 μg/ml (FIGS. 11A and 11B) in DMEM media with 1% FBS.

### Example 9

The objective of this study is to estimate the drug-like properties of the three cannabinoids in the ternary system, with the aim to determine the mode of administration, and optimal delivery methods. A combination of ADMET Predictor® and Poulin and Theil (2002) method were used to estimate the drug-like properties of CBD, CBC and CBN. An in-house, proprietary pharmacokinetic model was employed to simulate plasma and organ profiles.

TABLE 6
In silico estimation of the pharmacokinetic
parameters of CBD, CBC and CBN

| Pharmacokinetic | Cannabinoid | | | | | |
|---|---|---|---|---|---|---|
| parameters | CBD | | CBC | | CBN | |
| F¹ | 0.48 | | 0.12 | | 0.03 | |
| F_{g}² | 0.77 | | 0.6 | | 0.14 | |
| Fₗ³ | 0.62 | | 0.2 | | 0.21 | |
| Solubility, | 0.0004 | | 0.0001 | | 0.0002 | |
| mg/ml FSGF⁴ | | | | | | |
| Solubility, | 0.009 | | 0.003 | | 0.004 | |
| mg/ml FSIF⁵ | | | | | | |
| Solubility, | 0.12 | | 0.11 | | 0.12 | |
| mg/ml FeSIF⁶ | | | | | | |
| Cl_{TB}⁷, L/min | 0.709 | | 1.233 | | 1.206 | |
| Cl_{H}⁸, L/min | 0.726 | | 1.038 | | 1.035 | |
| Clᵣₑₙₐₗ⁹, L/min | 0 | | 0.195 | | 0.171 | |
| V_{d, ss}¹⁰ (L/kg) | 3.82 | | 3.66 | | 3.57 | |
| t_{1/2}¹¹, hours | 1.68 | | 0.64 | | 1.06 | |
| | V_{d}¹² | | V_{d}¹² | | V_{d}¹² | |
| | (L/kg) | Pₜₚ¹³ | (L/kg) | Pₜₚ¹³ | (L/kg) | Pₜₚ¹³ |
| Liver | 0.005 | 5.3 | 0.005 | 5.27 | 0.005 | 5.26 |
| Lung | 1.3 | 0.71 | 1.29 | 0.71 | 1.29 | 0.71 |
| Gut | 0.01 | 6.71 | 0.01 | 6.67 | 0.01 | 6.65 |
| Kidney | 0.14 | 3.2 | 0.14 | 3.18 | 0.14 | 3.17 |
| Muscle | 0.15 | 3.25 | 0.15 | 3.23 | 0.15 | 3.22 |
| Fat | 1.04 | 8.71 | 0.9 | 7.5 | 1.13 | 6.85 |

F¹: oral bioavailability; F_{g}²: fraction of dose absorbed from the gut; Fₗ³: absorbed fraction escaped the liver; FSGF⁴: fasted simulated gastric fluid; FSIF⁵: fasted simulated intestinal fluid; FeSIF⁶: fed simulated intestinal fluid; Cl_{TB}⁷: Total body clearance; Cl_{H}⁸: Hepatic clearance; Clᵣ⁹: renal clearance; V_{d, ss}¹⁰: Volume of distribution at steady state; t_{1/2}¹¹: Half-life; V_{d}¹²: Volume distribution of organ; Pₜₚ¹³: tissue/plasma partition coefficient (Poulin and Theil (2002)).

Table 6 is a summary of in silico estimations of the drug-like properties of CBD, CBC and CBN. Besides CBD, there is a paucity of pharmacokinetic data on CBC and CBN in humans. The total body clearance value of CBD estimated herein matches that reported in the literature (Meyer, Langos et al. 2018), providing qualitative support that this set of data can be used at a higher level.

CBD, CBC and CBN have extremely low solubility, and high gut and liver first-pass metabolism, making them poor candidates for oral delivery. These three cannabinoids are mainly cleared by the liver as indicated by their high Cl_{TB}, with values approaching that of hepatic blood flow (1.5 L/min).

Drugs with high first-pass metabolism have huge inter-individual variability, >10-fold variation is not uncommon. Intra-subject variability is also problem. These fluctuations in drug levels are issues when it comes to cancer treatment, as the effects of chemotherapeutic agents are highly non-specific, and hence, toxic. Dose titration is impractical. Furthermore, when it comes to multiple component formulas, like the ones disclosed in this invention, whose concentration ratios are crucial to eliciting synergistic effects, individual variations in pharmacokinetic profiles could pose a threat to therapeutic success.

The inter- and intra-subject variability can be circumvented by administering a candidate through vascular routes. AUC values of CBD varied around two-fold after intravenous injection (Meyer, Langos et al. 2018). This fluctuation is within the synergistic ratio range measured in this disclosure.

To better control the delivery and to maximize efficacy and minimize toxicity, a candidate can be infused intra-vascularly. Table 7 shows estimated infusion rates to achieve a steady state blood and liver level, which are equivalent to their respective IC₅₀ values.

TABLE 7
Estimation of in vivo intravascular infusion rates of
CBD, CBC/CBN and CBD/CBC/CBN with the aim to achieve respective
IC₅₀ values as steady state concentrations.

| | Component | | |
|---|---|---|---|
| Candidate | CBD | CBC | CBN |
| CBD | | | |
| C_{ss, liver}¹ ng/ml | 900 | n/a | n/a |
| K₀², mg/min | 1.23-3.86 | n/a | n/a |
| Daily Dose, mg | 1771.2-5558.4 | n/a | n/a |
| CBC/CBN | | | |
| C_{ss, liver}¹ ng/ml | n/a | 653.33 | 326.67 |
| K₀², mg/min | n/a | 1.83-2.32 | 0.77-1.08 |
| Daily Dose, mg | n/a | 2635.2-3340.8 | 1108.8-1555.2 |
| CBD/CBC/CBN | | | |
| C_{ss, liver}¹ ng/ml | 120 | 60 | 20 |
| K₀², mg/min | 0.16-0.51 | 0.17-0.21 | 0.05-0.07 |
| Daily Dose, mg | 230.4-734.4 | 244.8-302.4 | 72-100.8 |

C_{ss, liver}¹, steady state concentration in the liver;
K₀², intravascular infusion rate

Although in silico prediction of pharmacokinetic parameters has improved in the past decades, there are still rooms for improvement, in that, it is not uncommon to have predictions deviate by more than 5-fold from clinical values.

Prediction of clinical concentrations methods are less developed. A general approach is not yet available. In this disclosure, we shall derive physiological effective concentrations of the formulas described herein using that of a positive control which has both in vitro and in vivo data.

Based on in silico estimation nano-capsulation of the combination drug candidates may enhance the efficacy and reduce the toxicity of the candidates.

### REFERENCES

Banerjee, P., M. Dunkel, E. Kemmler and R. Preissner (2020). “SuperCYPsPred-a web server for the prediction of cytochrome activity.” Nucleic Acids Res 48(W1): W580-W585.
Blasco-Benito, S., M. Seijo-Vila, M. Caro-Villalobos, I. Tundidor, C. Andradas, E. Garcia-Taboada, J. Wade, S. Smith, M. Guzman, E. Perez-Gomez, M. Gordon and C. Sanchez (2018). “Appraising the “entourage effect”: Antitumor action of a pure cannabinoid versus a botanical drug preparation in preclinical models of breast cancer.” Biochem Pharmacol 157:285-293.
Calvi, L., D. Pentimalli, S. Panseri, L. Giupponi, F. Gelmini, G. Beretta, D. Vitali, M. Bruno, E. Zilio, R. Pavlovic and A. Giorgi (2018). “Comprehensive quality evaluation of medical Cannabis sativa L. inflorescence and macerated oils based on HS-SPME coupled to GC-MS and LC-HRMS (q-exactive orbitrap (R)) approach.” J Pharm Biomed Anal 150:208-219.
Chou, T. C. (2006). “Theoretical basis, experimental design, and computerized simulation of synergism and antagonism in drug combination studies.” Pharmacol Rev 58(3): 621-681.
Daemen, T., M. Velinova, J. Regts, M. de Jager, R. Kalicharan, J. Donga, J. J. van der Want and G. L. Scherphof (1997). “Different intrahepatic distribution of phosphatidylglycerol and phosphatidylserine liposomes in the rat.” Hepatology 26(2): 416-423.
Dai, Y. (2019). “Integration of systems biology and computer aided drug design technology n action mechanism of traditional chinese medicine.” Invest Clin 60(1): 207-214.
De Petrocellis, L., V. Vellani, A. Schiano-Moriello, P. Marini, P. C. Magherini, P. Orlando and V. Di Marzo (2008). “Plant-derived cannabinoids modulate the activity of transient receptor potential channels of ankyrin type-1 and melastatin type-8.” J Pharmacol Exp Ther 325(3): 1007-1015.
Geeleher, P., N. J. Cox and R. S. Huang (2014). “Clinical drug response can be predicted using baseline gene expression levels and in vitro drug sensitivity in cell lines.” Genome Biol 15(R47): 1-12.
Graziose, R., M. A. Lila and I. Raskin (2010). “Merging traditional Chinese medicine with modern drug discovery technologies to find novel drugs and functional foods.” Curr Drug Discov Technol 7(1): 2-12.
Hasegawa, H. (2004). “Proof of the mysterious efficacy of ginseng: basic and clinical trials: metabolic activation of ginsenoside: deglycosylation by intestinal bacteria and esterification with fatty acid.” J Pharmacol Sci 95(2): 153-157.
Javid, F. A., M. Duncan C. Stott (2018). Use of and Phytocannabinoids In The Treatment Of Ovarian Carcinoma. USPTO.
Jiang, M., C. Zhang, G. Zheng, H. Guo, L. Li, J. Yang, C. Lu, W. Jia and A. Lu (2012). “Traditional chinese medicine zheng in the era of evidence-based medicine: a literature analysis.” Evid Based Complement Alternat Med 2012:409568.
Kitisin, K., V. Packiam, J. Steel, A. Humar, T. C. Gamblin, D. A. Geller, J. W. Marsh and A. Tsung (2011). “Presentation and outcomes of hepatocellular carcinoma patients at a western centre.” HPB (Oxford) 13(10): 712-722.
Kleckner, A. S., I. R. Kleckner, C. S. Kamen, M. A. Tejani, M. C. Janelsins, G. R. Morrow and L. J. Peppone (2019). “Opportunities for cannabis in supportive care in cancer.” Ther Adv Med Oncol 11: 1758835919866362.
Koltai, H., P. Poulin and D. Namdar (2019). “Promoting cannabis products to pharmaceutical drugs.” Eur J Pharm Sci 132: 118-120.
Li, W. J., Y. W. Lian, Q. S. Guan, N. Li, W. J. Liang, W. X. Liu, Y. B. Huang, Y. Cheng and H. Luo (2018). “Liver-targeted delivery of liposome-encapsulated curcumol using galactosylated-stearate.” Exp Ther Med 16(2): 925-930.
Liang, K. L., R. S. Jiang, C. L. Lee, P. J. Chiang, J. S. Lin and Y. C. Su “Traditional (2012). Chinese Medicine ZHENG Identification Provides a Novel Stratification Approach in Patients with Allergic Rhinitis.” Evid Based Complement Alternat Med 2012: 480715.
Lorente, M., S. Torres, M. Salazar, A. Carracedo, S. Hernandez-Tiedra, F. Rodriguez-Fornes, E. Garcia-Taboada, B. Melendez, M. Mollejo, Y. Campos-Martin, S. A. Lakatosh, J. Barcia, M. Guzman and G. Velasco (2011). “Stimulation of the midkine/ALK axis renders glioma cells resistant to cannabinoid antitumoral action.” Cell Death Differ 18(6): 959-973.
Mayumi, K., T. Akazawa, T. Kanazu, S. Ohnishi and H. Hasegawa (2019). “Successful Prediction of Human Pharmacokinetics After Oral Administration by Optimized Physiologically Based Pharmacokinetics Approach and Permeation Assay Using Human Induced Pluripotent Stem Cell-Derived Intestinal Epithelial Cells.” J Pharm Sci.
Mayumi, K., S. Ohnishi and H. Hasegawa (2019). “Successful Prediction of Human Pharmacokinetics by Improving Calculation Processes of Physiologically Based Pharmacokinetic Approach.” J Pharm Sci 108(8): 2718-2727.
Mayumi, K., M. Tachibana, M. Yoshida, S. Ohnishi, T. Kanazu and H. Hasegawa (2020). “Successful Prediction of Human Pharmacokinetics by Physiologically-Based techniques using in vitro data.” J Pharm Sci TBA.
McKallip, R. J., M. Nagarkatti and P. S. Nagarkatti (2005). “Delta-9-tetrahydrocannabinol enhances breast cancer growth and metastasis by suppression of the antitumor immune response.” J Immunol 174(6): 3281-3289.
Meyer, P., M. Langos and R. Brenneisen (2018). “Human Pharmacokinetics and Adverse Effects of Pulmonary and Intravenous THC-CBD Formulations.” Medical Cannabis and Cannabinoids 1(1): 36-43.
Millar, S. A., N. L. Stone, A. S. Yates and S. E. O'Sullivan (2018). “A Systematic Review on the Pharmacokinetics of Cannabidiol in Humans.” Front Pharmacol 9:1365.
Moreno, E., M. Cavic, A. Krivokuca, V. Casado and E. Canela (2019). “The Endocannabinoid System as a Target in Cancer Diseases: Are We There Yet?” Front Pharmacol 10: 339.
Nagarkatti, P., R. Pandey, S. A. Rieder, V. L. Hegde and M. Nagarkatti (2009). “Cannabinoids as novel anti-inflammatory drugs.” Future Med Chem 1(7): 1333-1349.
Parolaro, D., P. Massi, A. Izzo, F. Borelli, G. Aviello, V. Di Marzo, L. De Petrocellis, A. S. Moriello, A. Ligresti, R. A. Ross, L. A. Ford, A.-G. S., M. Guzman, G. Velasco, M. Lorente, S. Torres, T. Kikuchi, G. Guy, C. Stott, S. Wright, A. Sutton, D. Potter and E. De Meijer (2013). Phytocannabinoids in the treatment of cancer. USPTO. USA, Otsuka Pharmaceutical Co., Ltd. GW Pharma Ltd.: 35.
Poulin, P. and F. P. Theil (2002). “Prediction of pharmacokinetics prior to in vivo studies. 1. Mechanism-based prediction of volume of distribution.” J Pharm Sci 91(1): 129-156.
Roberts, P. J. and C. J. Der (2007). “Targeting the Raf-MEK-ERK mitogen-activated protein kinase cascade for the treatment of cancer.” Oncogene 26(22): 3291-3310.
Russo, E. B. (2018). “The Case for the Entourage Effect and Conventional Breeding of Clinical Cannabis: No “Strain,” No Gain.” Front Plant Sci 9: 1969.
Sakellaropoulos, T., K. Vougas, S. Narang, F. Koinis, A. Kotsinas, A. Polyzos, T. J. Moss, S. Piha-Paul, H. Zhou, E. Kardala, E. Damianidou, L. G. Alexopoulos, I. Aifantis, P. A. Townsend, M. I. Panayiotidis, P. Sfikakis, J. Bartek, R. C. Fitzgerald, D. Thanos, K. R. Mills Shaw, R. Petty, A. Tsirigos and V. G. Gorgoulis (2019). “A Deep Learning Framework for Predicting Response to Therapy in Cancer.” Cell Rep 29(11): 3367-3373 e3364.
Shi, Q., H. Zhao, J. Chen, X. Ma, Y. Yang, C. Zheng and W. Wang (2012). “Study on TCM Syndrome Identification Modes of Coronary Heart Disease Based on Data Mining.” Evid Based Complement Alternat Med 2012: 697028.
Stott, C., M. Duncan and T. Hill (2017). Active pharmaceutical ingredient (API) comprising cannabinoids for use in the treatment of cancer. USPTO. USA, GW Pharma Limited: 18.
Su, S. B., W. Jia, A. Lu and S. Li (2014). “Evidence-Based ZHENG: A Traditional Chinese Medicine Syndrome 2013.” Evid Based Complement Alternat Med 2014: 484201.
Sun, S., J. Dai, W. Wang, H. Cao, J. Fang, Y. Y. Hu, S. Su and Y. Zhang (2012). “Metabonomic Evaluation of ZHENG Differentiation and Treatment by Fuzhenghuayu Tablet in Hepatitis-B-Caused Cirrhosis.” Evid Based Complement Alternat Med 2012: 453503.
Tam, Y. K., Y.-C. J. Lin, B. D. Sloely and C.-Y. Tseng (2019). Phytoestrogen product of red clover and pharmaceutical uses thereof. USPTO. USPTO. U.S.A, Sinoveda Canada Inc.
Tam, Y. K. and J. A. Tuszynski (2008). Pharmaceutical platform technology for the development of natural products P. C. Treaty.
Tekin, E., C. White, T. M. Kang, N. Singh, M. Cruz-Loya, R. Damoiseaux, V. M. Savage and P. J. Yeh (2018). “Prevalence and patterns of higher-order drug interactions in Escherichia coli.” NPJ Syst Biol Appl 4: 31.
Tomko, A. M., E. G. Whynot, L. D. Ellis and D. J. Dupre (2020). “Anti-Cancer Potential of Cannabinoids, Terpenes, and Flavonoids Present in Cannabis.” Cancers (Basel) 12(7).
Torres, S., M. Lorente, F. Rodriguez-Fornes, S. Hernandez-Tiedra, M. Salazar, E. Garcia-Taboada, J. Barcia, M. Guzman and G. Velasco (2011). “A combined preclinical therapy of cannabinoids and temozolomide against glioma.” Mol Cancer Ther 10(1): 90-103.
Tubaro, A., A. Giangaspero, S. Sosa, R. Negri, G. Grassi, S. Casano, R. Della Loggia and G. Appendino (2010). “Comparative topical anti-inflammatory activity of cannabinoids and cannabivarins.” Fitoterapia 81(7): 816-819.
Tyzack, J. D. and J. Kirchmair (2019). “Computational methods and tools to predict cytochrome P450 metabolism for drug discovery.” Chem Biol Drug Des 93(4): 377-386.
Urits, I., M. Borchart, M. Hasegawa, J. Kochanski, V. Orhurhu and O. Viswanath (2019). “An Update of Current Cannabis-Based Pharmaceuticals in Pain Medicine.” Pain Ther.
Vara, D., M. Salazar, N. Olea-Herrero, M. Guzman, G. Velasco and I. Diaz-Laviada (2011). “Anti-tumoral action of cannabinoids on hepatocellular carcinoma: role of AMPK-dependent activation of autophagy.” Cell Death Differ 18(7): 1099-1111.
Walter, L. and N. Stella (2004). “Cannabinoids and neuroinflammation.” Br J Pharmacol 141(5): 775-785.
Wang, W. Y., H. Zhou, Y. F. Wang, B. S. Sang and L. Liu (2021). “Current Policies and Measures on the Development of Traditional Chinese Medicine in China.” Pharmacol Res 163: 105187.
Wang, X., H. Sun, A. Zhang, G. Jiao, W. Sun and Y. Yuan (2011). “Pharmacokinetics screening for multi-components absorbed in the rat plasma after oral administration traditional Chinese medicine formula Yin-Chen-Hao-Tang ultra by performance liquid chromatography-electrospray ionization/quadrupole-time-of-flight mass spectrometry combined with pattern recognition methods.” Analyst 136(23): 5068-5076.
Weathers, P. J., M. A. Elfawal, M. J. Towler, G. K. Acquaah-Mensah and S. M. Rich (2014). “Pharmacokinetics of artemisinin delivered by oral consumption of Artemisia annua dried leaves in healthy vs. Plasmodium chabaudi-infected mice.” J Ethnopharmacol 153(3): 732-736.
Weathers, P. J., M. Towler, A. Hassanali, P. Lutgen and P. O. Engeu (2014). “Dried-leaf Artemisia annua: A practical malaria therapeutic for developing countries?” World J Pharmacol 3(4): 39-55.
Zhang, G. B., Q. Y. Li, Q. L. Chen and S. B. Su (2013). “Network pharmacology: a new approach for chinese herbal medicine research.” Evid Based Complement Alternat Med 2013:621423.
Zhao, C., H. Liu, P. Miao, H. Wang, H. Yu, C. Wang and Z. Li (2019). “A Strategy for Selecting “Q-Markers” of Chinese Medical Preparation via Components Transfer Process Analysis with Application to the Quality Control of Shengmai Injection.” Molecules 24(9).
Zhong, N. (2020). Cannabinol inhibits proliferation and induces cell cycle arrest and apotosis in glioblastoma, hepatocellular carcinoma and breast cancer cells. M. Sc., University of Lethbridge.

1-32. (canceled)

33. A method to efficiently identify an optimal composition comprising active compounds from an herb or herbal formula for treating a disease, said method comprising:
1) Obtaining chemical profile in an herb or herbal formula from existing databases or producing chemical profile using high resolution mass spectrometry if no records are found in the existing databases;
2) Identifying and developing appropriate in vitro models that have clinical relevance to said disease;
3) Computationally identifying, from the chemical profile obtained 1), potential active ingredients as primary candidate compounds using methods from systems biology, systems pharmacology, bioinformatics and machine learning based approaches based on three criteria,
a. Their efficacies in treating the disease;
b. Their influence on the absorption and metabolism of potential active ingredients;
c. Their drug-like properties and their metabolites;
4) Screening the primary candidate compounds obtained in step 3) by following a set of predetermined and adjustable pharmacodynamic and pharmacokinetic criteria and four iterative procedures,
a. Evaluating pair-wise interactions of primary candidate compounds based on a pairwise-based experimental approach using in vitro models developed in step 2), resulting in pairs of compounds synergistically/antagonistically regulate the disease, and
b. Predicting multi-compound synergism to treat the disease using the results obtained in step 4a and a cluster expansion method, resulting in a list of secondary candidate compounds with specific concentration ratios, and
c. Validating and testing the secondary candidate compounds obtained in step 4b for efficacy and side effects in vitro, and
d. Repeating step and 4c) 4b) until step 4b) is validated, leading to a list of active compounds; and
5) Formulating an optimal composition comprising the active compounds in view of compound-compound interactions, wherein the composition possesses maximum efficacy with minimum side effects in treating the disease.

34. The method of claim 33, wherein the compound-compound interactions comprise pair-wise interactions; or wherein the compound-compound interactions comprise higher order interactions.

35. The method of claim 34, wherein the pair-wise interactions are quantified by two-dimensional arrays based on an appropriate in vitro model.

36. The method of claim 34, wherein the higher order interactions are modelled by using a Cluster expansion validated with experimental inputs.

37. The method of claim 33, wherein the drug-like properties are estimated using parameters generated in vitro and in silico physiologically based pharmacokinetic models.

38. The method of claim 37, wherein the drug-like properties generated in vitro and in silico are used to estimate in vivo pharmacokinetic parameters with proper scaling using one or more of the followings:
1) human intestinal microsomes;
2) 3D intestinal model;
3) simulated gastric or intestinal fluids;
4) established anerobic method;
5) human liver microsomes;
6) S-9 fraction;
7) hepatocytes;
8) 3D model of a human liver;
9) 2D kidney model; and
10) 3D model organoid model.

39. The method of claim 33, wherein the drug-like properties are estimated using existing algorithms, commercial or open-source software, wherein the chemical profile in the herb or herb formula comprises compounds that could induce or inhibit metabolism of or alter the transport of the potential active ingredients and the metabolites, wherein the chemical profile in the herb or herb formula comprises compounds that could increase or decrease efficacies or side effects associated with one or more of the potential active ingredients and the metabolites in treating the disease, wherein the composition is formulated in view of compound-compound interactions established using in vitro methods, wherein the composition is formulated for easy or efficient delivery, wherein the composition is formulated as tablets, solutions, suspensions, creams, emulsions, or nano encapsulated emulsions, wherein the composition is formulated for oral, sublingual, topical, subcutaneous, intramuscular, intravenous or intraarterial administration, or wherein the disease is selected from the group consisting of cancer, diabetes, cardiovascular, hepatic, renal, lung, neurodegenerative, arthritic, immune, auto-immune diseases.

40. An optimal composition comprising one or more of CBD, CBC and CBN, for treating hepatocellular carcinoma.

41. The composition of claim 40, wherein said composition comprises cannabichromene (CBC) and cannabinol (CBN) with optimal ratios; or wherein said composition comprises CBC, CBN and CBD with optimal ratios.

42. The composition of claim 41, wherein said composition comprise CBC and CBN with a weight ratio ranging from 16:1 to 1:2.

43. The composition of claim 41, wherein the ratios of CBD:CBC:CBN range from 1:1:1 to 9:3:1.

44. A phytochemical composition comprising one or more nano-encapsulated cannabinoids for treating hepatocellular carcinoma.

45. The composition of claim 44, wherein said cannabinoids are selected from the group consisting of cannabidiol (CBD), cannabichromene (CBC) and cannabinol (CBN).

46. The composition of claim 45, wherein said composition comprises: a) nano-encapsulated CBD; b) nano-encapsulated CBC/CBN; or c) nano-encapsulated CBD/CBC/CBN.

47. The composition of claim 44, wherein the composition comprises nano-encapsulated CBD at a dosage less than half of that of the non-encapsulated form.

48. A method of treating hepatocellular carcinoma by administering the composition of claim 41 into a subject in need thereof.

49. The method of claim 48, wherein the composition is administered via intravascular or subcutaneous injection; or wherein the composition is selectively distributed to liver cancer cells, which allows maximization of efficacy and minimization of toxicity.

50. The method of claim 48, wherein the method demonstrates a comparable or higher efficacy in comparison to sorafenib.
